(19) United States
(12) Patent Application Publication
(10) Pub. No.: US 2026/0232624 A1
(43) Pub. Date: Aug. 13, 2026

(54) Polyphenol-Containing Compositions for Upregulating Camp Gene Expression

(71) Applicant: The Board of Trustees of the Leland Stanford Junior University, Stanford, CA (US)

(72) Inventors: John A. Fortkort, Austin, TX (US); Annelise E. Barron, Palo Alto, CA (US)

(21) Appl. No.: 19/474,611

(22) PCT Filed: Apr. 12, 2024
(86) PCT No.: PCT/US2024/024474

§ 371 Date: Oct. 10, 2025

Related U.S. Application Data
(60) Provisional application No. 63/496,292, filed on Apr. 14, 2023.

(51) Int. Cl. A61K 31/352; A61K 31/05; A61K 31/09; A61K 31/19; A61K 31/353; A61K 31/417; A61P 1/00; G01N 33/68

(52) U.S. Cl. CPC A61K 31/352; A61K 31/05; A61K 31/09; A61K 31/19; A61K 31/353; A61K 31/417; A61P 1/00; G01N 33/6893; G01N 2800/06

(57) ABSTRACT

A method is for modulating tight junction (TJ) integrity in a subject. The method includes (a) assessing TJ integrity in the subject by quantifying at least one biomarker of TJ integrity in the subject; (b) administering to the subject a first composition containing (i) at least one polyphenol, and (ii) a second substance which is not a polyphenol and which upregulates CAMP gene expression in the subject; (c) reassessing TJ integrity in the subject by quantifying at least one biomarker of TJ integrity in the subject; and (d) repeating steps (b) and (c) until the value of said at least one biomarker is within a target range.

### CROSS-REFERENCE TO RELATED APPLICATIONS

The present application claims the benefit of priority from U.S. Provisional Application No. 63/496,292, filed on Apr. 14, 2023, which has the same title and the same inventors, and which is incorporated herein by reference in its entirety.

### FIELD OF THE DISCLOSURE

The present application relates generally to compositions and methodologies for modulating tight junctions and the corresponding effectiveness of epithelial barriers incorporating them, and more specifically to methods for treating or preventing diseases by modulating such epithelial barriers with compositions comprising at least one polyphenol and at least one substance which upregulates CAMP gene expression.

### BACKGROUND OF THE DISCLOSURE

Human cathelicidin LL-37 is centrally important to human host defense. The significance and efficacy of this peptide is attested to by the fact that it has been evolutionally conserved for over 300 million years. LL-37 is unique in both the human proteome and within primates. The expression of LL-37 is uniquely vitamin D3 dependent within humans, monkeys, and apes. A retinoid (or other RXRα agonist) such as vitamin A or dodecahexanoic acid (DHA) is also obligate for LL-37 expression.

Various substances are known which upregulate the CAMP gene that encodes LL-37. For example, vitamin D, phenyl-butyrate (PBA) and the benzamide histone deacetylase inhibitor Entinostat are all inducers of the CAMP gene. In the case of Entinostat, this occurs via activation of STAT3 and HIF-1α transcription factors. See [Miraglia, E., Nylén, F., Johansson, K. et al. Entinostat up-regulates the CAMP gene encoding LL-37 via activation of STAT3 and HIF-1α transcription factors. Sci Rep 6, 33274 (2016). https://doi.org/10.1038/srep33274; the disclosure of which is hereby incorporated by reference in its entirety].

### BRIEF DESCRIPTION OF THE DRAWINGS

FIGS. 1-2 depict the structure and composition of tight junction (TJ) barriers.

FIG. 3 depicts the chemical structure of quercetin.

### SUMMARY OF THE DISCLOSURE

In one aspect, a method is provided for modulating tight junction (TJ) integrity in a subject. The method comprises (a) assessing TJ integrity in the subject by quantifying at least one biomarker of TJ integrity in the subject; (b) administering to the subject a composition containing (i) at least one polyphenol, and (ii) a second substance which is not a polyphenol and which upregulates CAMP gene expression in the subject; (c) reassessing TJ integrity in the subject by quantifying at least one biomarker of TJ integrity in the subject; and (d) while the value of said at least one biomarker is outside of a target range, repeating steps (b) and (c).

In another aspect, a method for attenuating dysbiosis in a subject, comprising (a) assessing a state of dysbiosis in a subject by quantifying at least one dysbiosis biomarker in the subject; (b) administering to the subject a composition containing (i) at least one polyphenol, and (ii) a second substance which is not a polyphenol and which upregulates CAMP gene expression in the subject; and (c) repeating steps (a) and (b) until the value of said at least one dysbiosis biomarker is within a target range.

In a further aspect, a method is provided for determining whether an individual has an abnormal level of zonulin in their blood serum. The method comprises (a) ascertaining blood serum levels (Z_{bs}) of zonulin in the individual; (b) comparing the ascertained Z_{bs} to blood serum levels of zonulin in a control group (Z_{c}) comprising healthy, age-matched or sex-matched individuals, thereby determining Δ_{bs}=|Z_{bs}−Z_{c}|, wherein the control group may include relatives of the individual; (c) administering a composition comprising a polyphenol and a material which induces CAMP gene expression to the individual; and (d) repeating steps a-c until Δ_{bs} is within a predetermined range.

In yet another aspect, a method is provided for treating an individual with an abnormal level of zonulin in their blood serum. The method comprises monitoring the blood serum levels of zonulin in the individual; and administering to the individual, during the monitoring, a composition comprising a polyphenol and a material which induces CAMP gene expression to the individual until the difference between blood serum levels of zonulin in the subject and those of a reference control are not statistically significant.

In still another aspect, a method is provided for assessing the integrity of tight junction (TJ) barriers in a subject. The method comprises measuring the presence, concentration or amount of at least one biomarker in a biological specimen obtained from the subject, wherein the at least one biomarker correlates with TJ barrier integrity; and comparing the measured presence, concentration or amount of the at least one biomarker to a reference value to determine the TJ barrier integrity of the subject.

In a further aspect, a method of treating a subject is provided. The method comprises (a) ascertaining a microbiota index measured in a sample of subgingival fluid from the individual; (b) comparing the microbiota index to a reference value determined through statistical analysis or machine learning of the preponderance of microbial genera or species in subgingival fluid from healthy and diseased subjects; (c) determining the degree of oral dysbiosis in the individual based on the comparison; and (d) administering a composition to the subject until the degree of oral dysbiosis is within a predetermined range; wherein the composition comprises a polyphenol and a material which induces CAMP gene expression in the individual.

In another aspect, a method of treating a subject is provided which comprises (a) ascertaining a microbiota index measured in a sample taken from a source microbiome selected from the group consisting of the gut, skin, urinary, aural, ocular, genital, pulmonary, nasopharyngeal, tonsillar, and umbilical microbiomes; (b) comparing the microbiota index to a reference value determined through statistical analysis or machine learning of the preponderance of microbial genera or species in a sample from healthy and diseased subjects in the source microbiome; (c) determining the degree of dysbiosis in the source microbiome based on the comparison; and (d) administering a composition to the subject until the degree of dysbiosis in the source microbiome is within a predetermined range; wherein the composition comprises a polyphenol and a material which induces CAMP gene expression in the individual.

### DETAILED DESCRIPTION

As used herein, the following terms have the noted definitions.

“Polyphenol” refers to a composition having a polyphenol structure (that is, having multiple hydroxyl groups on an aromatic ring). The term includes compositions having a polyphenol structure which are selected from the group consisting of phenolic acids, flavonoids, stilbenes, lignans, tannins, curcuminoids (e.g., curcumin), ellagitannins, xanthones (e.g., mangostin) and derivatives of the foregoing.

“Phenolic acid” refers to a substance containing a phenolic ring and having an organic carboxylic acid moiety function on the C6-C1 skeleton. The term includes hydroxybenzoic acids and hydroxycinnamic acids. Specific, nonlimiting examples of phenolic acids include caffeic acid, salicylic acid, hydroxybenzoic acids (e.g., gallic acid), hydroxycinnamic acids (e.g., caffeic acid, ferulic acid).

“Flavonoids” refers to a class of compositions that includes flavones (e.g., apigenin, luteolin), flavonols (e.g., quercetin, kaempferol), flavanols (e.g., catechins, epicatechin), flavanones (e.g., hesperidin, naringenin), isoflavones, proanthocyanidins, and anthocyanins (e.g., cyanidin, delphinidin).

“Isoflavanoids” refers to a class of flavonoid phenolic compounds derived from 3-phenylchromen-4-one (3-phenyl-1,4-benzopyrone) structure. Specific, nonlimiting examples of isoflavanoids include genistein and daidzein.

“Homoisoflavonoids” refers to a class of phenolic compounds having a 3-benzylidenechroman-4-one structure. Specific, nonlimiting examples of homoisoflavonoids include portulacanones A, B, C and D; sappanol, sappanone A, episappanol, 3′-deoxysappanol, 3′-O-methylsappanol and 3′-O-methylepisappanol; and scillavones A and B.

“Neoflavanoids” refers to a class of flavonoid phenolic compounds derived from a 4-phenylcoumarine (4-phenyl-1,2-benzopyrone) structure.

“Stilbenes” refers to 1,2-diphenylethene and its stereoisomers, including the cis isomer (Z)-stilbene and the trans isomer €-stilbene.

“Lignans” refers to polyphenols derived from phenylalanine. Some specific, nonlimiting examples of lignans include justicidin A, matairesinol, pinoresinol, podophyllotoxin, secoisolariciresinol, steganacin, enterolactone, enterodiol.

Proanthocyanidins (PACs) are a subclass of flavonoid. Cranberry PACs may be especially useful in some of the compositions and methodologies disclosed herein. PACs in cranberries are predominantly composed of A-type linkages, which are more resistant to degradation and have been shown to have stronger biological activities than B-type PACs. The PACs found in cranberries are oligomers and polymers of flavan-3-ols, primarily (−)-epicatechin and (+)-catechin. Cranberry PACs are typically classified into two categories: low-molecular-weight PACs and high-molecular-weight PACs. Low-molecular-weight PACs are composed of monomers and dimers, while high-molecular-weight PACs are composed of oligomers and polymers. Both low- and high-molecular-weight PACs are present in cranberries.

“Stilbenoids” refers to hydroxylated derivatives of stilbene. Some specific, non-limiting examples of stilbenoids include aglycones such as resveratrol, piceatannol, pinosylvin and pterostilbene, and glycosides such as astringin and piceid; resveratrol dimers such as, for example, ampelopsin A and ampelopsin B. The term also includes (E)-3,5-dihydroxy-4-isopropyl-trans-stilbene and 2-isopropyl-5-[(E)-2-phenylvinyl]benzene-1,3-diol.

“Tannins” (or tannoids) refers to a class of polyphenolic biomolecules that includes, but is not limited to, tannic acid (including the forms of tannic acid known as quercitannic acid, ellagic acid, certain proanthocyanidins, and gallotannic acid).

The antimicrobial properties of LL-37 have been the subject of considerable research and are discussed, for example, in U.S. US2019/0015361 (Barron et al.), entitled “Polytherapy Modulating Cathelicidin Gene Expression Modulation For The Treatment Of Alzheimer's Disease And Other Conditions”, and WO2021188836 (Barron et al.), entitled “Upregulation Of Cathelicidin Gene Expression As An Adjuvant To Other Treatments For Diseases”, both of which are incorporated herein by reference in their entirety. However, the role of LL-37 on other aspects of human health has not been fully appreciated.

It has now been found that proper CAMP gene expression (i.e., proper regulation of LL-37) is crucial to maintaining, restoring or preventing the disruption of the integrity of tight junction (TJ) barriers throughout the body including, for example, those in the blood-brain barrier (BBB) and the epithelial layers existing in the skin, bladder, eyes, colon, gut and intestines. It has further been found that other classes of materials, especially polyphenols, have similar or other beneficial effects on TJ barriers, and can work (often synergistically) with LL-37 in maintaining, restoring or preventing the disruption of the integrity of these barriers. It has also been found that disruption of TJ barriers is critical to the pathology of a wide variety of diseases. Indeed, some disease pathologies implicate the disruption of two or more distinct TJ barriers in different parts of the body, such as those in the gut epithelia and those in the BBB. Hence, maintaining, restoring or preventing the disruption of the integrity of TJ barriers is a potentially powerful aspect of human health and longevity.

There is thus a need in the art for systems and methodologies for assessing or quantifying TJ barrier integrity in subjects. There is further a need in the art for such systems and methodologies which can be readily and rapidly performed and are thus suitable for daily or regular use. There is further a need in the art for therapeutic feedback tools that may be utilized to perform such assessments, to determine when the integrity of TJ barriers is outside of a target range and requires remediation, and to determine when remediation of TJ barriers has been achieved.

It has also been found that mere upregulation of CAMP gene expression, without more, may be insufficient to achieve desired therapeutic or salutary effects and, in some cases, may even be harmful. For example, expression of the CAMP gene is induced in keratinocytes during inflammatory disorders. [Frohm M, Agerberth B, Ahangari G, Stahle-Backdahl M, Lidén S, Wigzell H, Gudmundsson GH. The expression of the gene coding for the antibacterial peptide LL-37 is induced in human keratinocytes during inflammatory disorders. J Biol Chem. 1997 Jun. 13; 272(24):15258-63. doi: 10.1074/jbc.272.24.15258. PMID: 9182550; the disclosure of which is hereby incorporated by reference in its entirety]. Similarly, CAMP gene expression has also been associated with certain autoimmune diseases such as experimental autoimmune encephalomyelitis (EAE), multiple sclerosis (MS) and psoriasis. [Smith K J, Minns D, McHugh B J, Holloway R K, O'Connor R, et al. (2022) The antimicrobial peptide cathelicidin drives development of experimental autoimmune encephalomyelitis in mice by affecting Th17 differentiation. PLOS Biology 20(8): e3001554. https://doi.org/10.1371/journal.pbio.3001554; the disclosure of which is hereby incorporated by reference in its entirety]. Without wishing to be bound by theory, this is believed to result from the ability, in some cases, for CAMP gene expression to induce the production of proinflammatory cytokines. There is thus a need in the art for a means of mitigating the possible adverse effects of CAMP gene expression, and in particular, the possible proinflammatory responses that may be attendant thereto.

It has also been found that microbiota dysbiosis, especially oral (e.g., subgingival) and gut dysbiosis, is an important aspect of many diseases. [Hou, K., Wu, Z X., Chen, X Y. et al. Microbiota in health and diseases. Sig Transduct Target Ther 7, 135 (2022). https://doi.org/10.1038/s41392-022-00974-4; the disclosure of which is hereby incorporated by reference in its entirety]. In some diseases, microbiota dysbiosis occurs in conjunction with inflammation. For example, periodontitis is characterized by an inflammatory host response caused by the gingival expression of inflammatory cytokines. [Cekici A, Kantarci A, Hasturk H, Van Dyke T E. Inflammatory and immune pathways in the pathogenesis of periodontal disease. Periodontol 2000. 2014 February; 64(1):57-80. doi: 10.1111/prd.12002. PMID: 24320956; PMCID: PMC4500791; the disclosure of which is hereby incorporated by reference in its entirety]. The microenvironment created by such inflammation may be conducive to the growth of pathogenic bacteria such as Enterococcus, Neisseria and Pseudomonas, which may displace symbiotic microbiota that are associated with good health. [Vieira Colombo A P, Magalhaes C B, Hartenbach F A, Martins do Souto R, Maciel da Silva-Boghossian C. Periodontal-disease-associated biofilm: A reservoir for pathogens of medical importance. Microb Pathog. 2016 May; 94:27-34. doi: 10.1016/j.micpath.2015.09.009. Epub 2015 Sep. 28. PMID: 26416306; the disclosure of which is hereby incorporated by reference in its entirety]. Hence, effective treatment of periodontitis and other oral diseases requires not only attenuation of the inflammation common to such diseases, but also rectification of the dysbiosis of microbiota that may occur as a result of inflammation. There is thus a need in the art for methods, compositions and systems for treating diseases such as periodontitis which can alleviate inflammation while simultaneously rectifying the dysbiosis of microbiota, especially oral and/or gut dysbiosis.

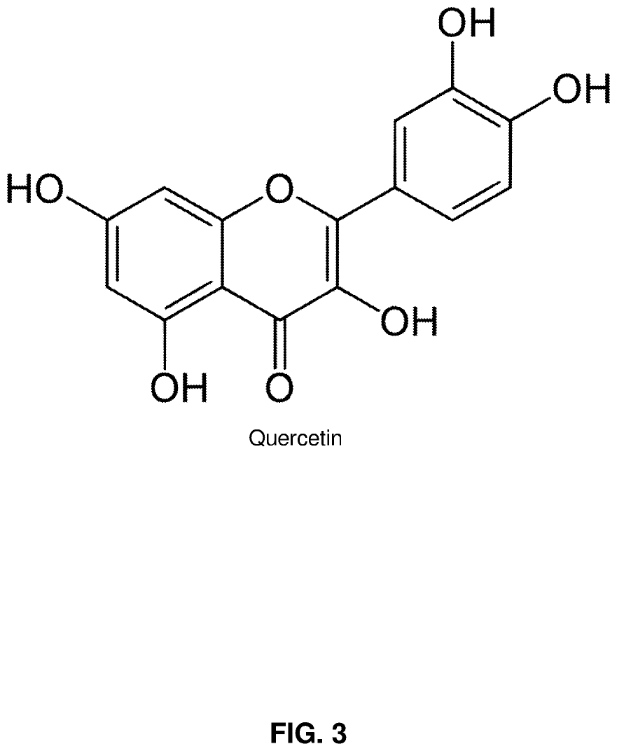

The foregoing needs may be met by the systems, methodologies and compositions described herein. In some embodiments, a pharmaceutical composition or dietary supplement is provided which comprises a first composition (such as, for example, vitamin D3) which induces CAMP gene expression in a subject, and a second composition (which is preferably a polyphenol such as, for example, quercetin (see FIG. 3)) which downregulates one or more proinflammatory cytokines. The second composition also preferably promotes tight junction (TJ) layer (including BBB) integrity. Without wishing to be bound by theory, it is believed that the second composition may act to suppress part or all of any inflammatory profile of the first substance (and in particular, all or part of any upregulation of certain proinflammatory cytokines which contribute to the inflammatory profile), thus providing the benefits of CAMP gene expression while suppressing some or all of its potentially deleterious effects. Through proper selection of the second composition (for example, through the use of a second composition containing quercetin (see FIG. 3), the green tea polyphenol epigallocatechin-3-gallate, or cranberry proanthocyanidins), the dysbiosis associated with many disease states may be remedied. For example, through proper selection of the second composition, oral dysbiosis associated with periodontitis may be remedied by reducing inflammation and promoting a microenvironment in the oral cavity which is conducive to maintaining a salutary symbiotic microbiota.

Some of the embodiments of the systems and methodologies disclosed herein may feature a feedback loop for assessing TJ barrier integrity or the degree of microbiota dysbiosis and using the results to inform subsequent treatment steps. In some such embodiments, the integrity of one or more tight junction (TJ) barriers or the degree of microbiota dysbiosis in a subject is determined (and preferably quantified) by ascertaining the presence and/or concentration or amount of at least one biomarker in a biological specimen present in, or taken from, the subject, wherein the at least one biomarker correlates with TJ barrier integrity or with microbiota dysbiosis.

For example, blood serum levels of the protein zonulin are found to correlate strongly with intestinal permeability. Hence, if the at least one biomarker is the protein zonulin, then its presence in, for example, a sample of blood serum taken from the subject may be quantified using an appropriate ELISA test. See, e.g., [Fasano A. Zonulin, regulation of tight junctions, and autoimmune diseases. Ann N Y Acad Sci. 2012 July; 1258(1):25-33. doi: 10.1111/j.1749-6632.2012.06538.x. PMID: 22731712; PMCID: PMC3384703], which is incorporated herein by reference in its entirety. If the ELISA test indicates that the integrity of a TJ barrier in the subject is outside of an acceptable range (for example, if blood serum levels of zonulin are a standard deviation above a reference value, and preferably 2 standard deviations above the reference value), a composition may be administered to the subject which contains a polyphenol (such as, for example, quercetin) and a material which induces CAMP gene expression (such as, for example, vitamin D3). As discussed in greater detail below, various reference values may be utilized for these purposes, although the use of suitable statistics (e.g., mean, variance and standard deviations) taken from a suitable reference population are preferred. The ELISA test may be performed again periodically until it indicates that integrity of the TJ barrier layer has been restored to a satisfactory level. Thereafter, if desired, the composition may be administered to the subject on a regular interval to maintain a suitable level of TJ barrier layer integrity. In some embodiments, the composition administered to the subject may change depending on the results of the feedback loop.

As an illustration of the foregoing, blood serum levels of zonulin may be ascertained for an individual and compared to the blood serum levels of zonulin in healthy, aged-matched or sex-matched controls. In some cases, the controls may be relatives of the individual. For example, the individual may exhibit serum zonulin levels of 2.37±0.17 ng/mg protein, compared to relatives (1.75±0.27 ng/mg protein, P=0.05) and control subjects (0.31±0.03 ng/mg protein, P<0.00001). A remedial composition may be administered to the subject (for example, a composition containing a polyphenol (such as, for example, quercetin) and a material which induces CAMP gene expression (such as, for example, vitamin D3)) until the difference between blood serum levels of zonulin in the subject and those of the control(s) are not statistically significant. The difference may be determined not to be statistically significant if, for example, it is less than two standard deviations, or less than one standard deviation, from the mean of the control group.

As a further example, if the at least one biomarker is a microbiota index measured, for example, in a sample of subgingival fluid, then the degree of oral dysbiosis in the subject may be ascertained by comparing the index to a reference value. The reference value may be, for example, a vector or scalar value whose value is ascertained through statistical analysis or by applying machine learning to the measurement of the preponderance of microbial genera or species in subgingival fluid from healthy and diseased subjects.

A particular, non-limiting example of how such an index may be computed and utilized to ascertain oral dysbiosis may be found, for example, in [Chen T, Marsh P D, Al-Hebshi N N. SMDI: An Index for Measuring Subgingival Microbial Dysbiosis. J Dent Res. 2022 March; 101(3):331-338. doi: 10.1177/00220345211035775. Epub 2021 Aug. 25. PMID: 34428955; PMCID: PMC8982011], which is incorporated herein by reference in its entirety. A similar technique may be utilized to compute microbiota indices and utilize them to ascertain dysbiosis in various other places or microbiomes in the body including, for example, in the gut, skin, bladder, ears, eyes, genitals, lungs, nose, tonsils, umbilicus, and structures surrounding the foregoing tissues for example, surrounding structures of the eye can include (but are not limited to) a conjunctiva, eyelids, and tear ducts).

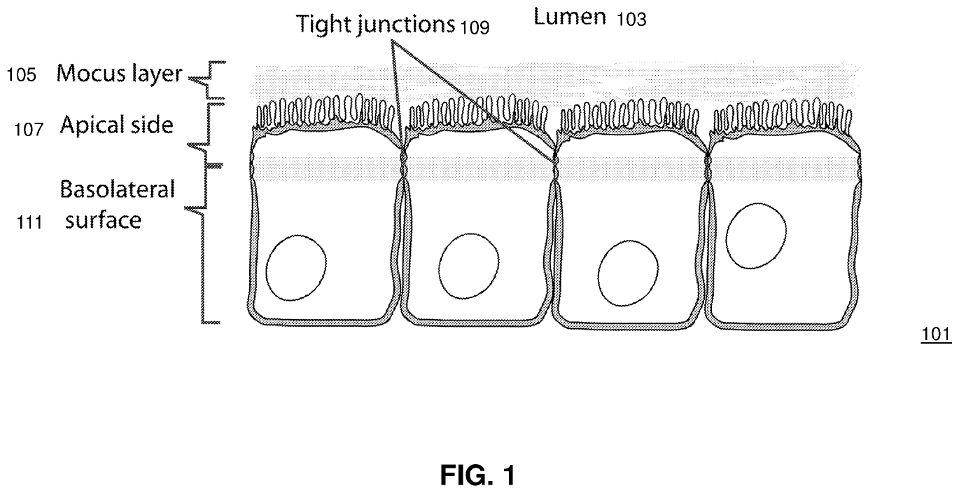

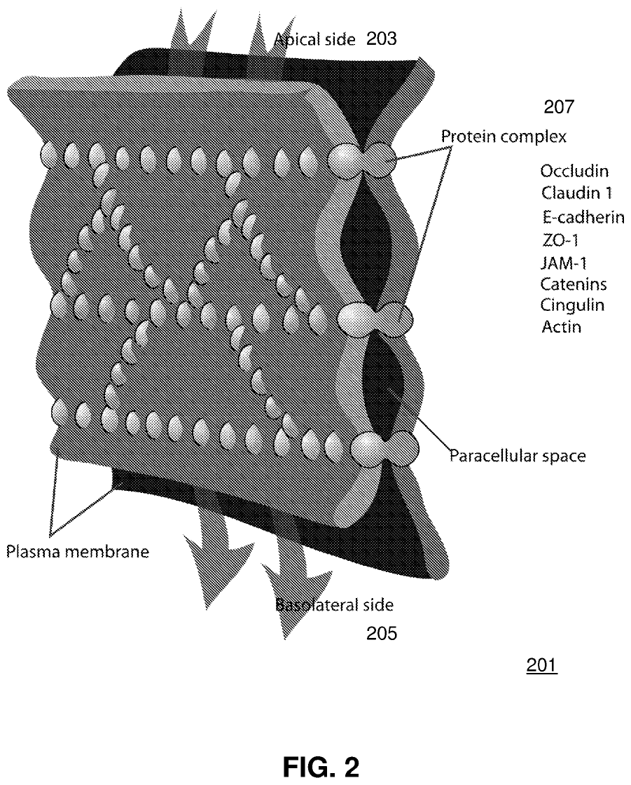

The systems, compositions and methodologies disclosed herein may be further understood with respect to FIGS. 1-2, which illustrate a typical TJ layer. As seen therein, TJs are multiprotein junctional complexes which function is to prevent leakage of solutes and water, and which provide a seal between the epithelial cells. Tight junctions may also serve as leaky pathways by forming selective channels for small cations, anions, or water.

FIG. 1 is a cross-sectional view of an epithelial cell layer 101, highlighting some of the key structural and functional components thereof. At the top of the figure, the lumen 103 is a hollow cavity or the interior space of a tubular structure. Adjacent to the lumen 103 is the mucus layer 105, depicted as a viscous protective coating that covers the apical side 107 of the epithelial cells, facing the lumen 103. This mucus layer 105 functions to trap pathogens and particulate matter, protecting the underlying cells.

The apical side 107 of the epithelial cells 101, directly beneath the mucus layer 105, may contain specialized structures such as microvilli, enhancing the surface area for absorption or secretion. The tight junctions 109 are prominently featured just below the apical surface, encircling each cell and serving as a critical barrier that regulates the passage of substances between the cells, thus maintaining the cellular polarity and integrity of the epithelial layer.

Beneath the tight junctions 109, the cells' basolateral surface 111 interfaces with the underlying tissue and is involved in cell communication and adhesion. The basolateral surface 111 may include structures such as lateral and basal infoldings and connections to the extracellular matrix and neighboring cells, highlighting its role in maintaining tissue structure and function.

FIG. 2 depicts an epithelial cell boundary 201, emphasizing the organization of the plasma membrane and associated protein complexes. The apical side 203 of the epithelial cell boundary 201 is depicted at the top, facing the lumen or external environment, and the basolateral surface 205 is at the bottom, interfacing with internal tissues.

The plasma membrane envelops the cell, with distinct compositions on the apical 203 and basolateral 205 sides to maintain cell polarity. Highlighted within the plasma membrane are various protein complexes 207 critical for cellular function and integrity. The protein complexes 207 are shown interlocking adjacent cells, thereby sealing the paracellular space to prevent the leakage of solutes and water. These include the tight junction proteins occludin, claudin-1, and ZO-1. Junctional Adhesion Molecule (JAM-1) is a protein disposed near the tight junctions which plays a role in cell adhesion and intracellular signaling. Adherens junctions are disposed just below the tight junctions, featuring E-cadherin and associated catenins. These proteins facilitate cell-to-cell adhesion and link to the actin cytoskeleton, providing mechanical stability to the tissue. The additional cytoskeletal and linking proteins cingulin and actin are shown within the junctional complexes. These proteins support the junctional structure and link the membrane proteins to the cell's internal cytoskeleton. The paracellular space 207 between the cells is a controlled environment that governs the passage of substances using the tight and adherens junctions. The basolateral surface 205 is typically equipped with receptors and other molecular structures that communicate with the internal environment, maintaining the cell's physiological functions and interactions with the extracellular matrix and neighboring cells

TJs are composed of branched networks of independently acting sealing strands in which the efficiency of the TJ in restricting ion passage increases exponentially as a function of the number of strands. Each strand is formed from a row of transmembrane proteins that are embedded in both plasma membranes such that the extracellular domains join one another directly.

TJs consist of a variety of transmembrane and cytoplasmic proteins. The three major transmembrane proteins are occludins, claudins, and junction adhesion molecule (JAM) proteins. These associate with different peripheral membrane proteins such as ZO-1 which are located on the intracellular side of the plasma membrane, and which anchor the strands to the actin component of the cytoskeleton. Consequently, TJs serve as connectors between the cytoskeletons of adjacent cells.

Occludin consists of four transmembrane domains in which the N-terminus and the C-terminus of the protein are both intracellular. Occludin forms two extracellular loops and one intracellular loop which serve to regulate paracellular permeability. Occludin also plays significant roles in cellular structure and barrier function.

Claudins have four transmembrane domains and a loop structure which is similar to that of occludin. Claudins act as the backbone of TJs and play a key role in the ability of TJs to seal the paracellular space.

Junctional Adhesion Molecules (JAMs) have a single transmembrane domain. JAMs aid in the regulation of the paracellular pathway function of tight junctions and also help to maintain cell polarity.

Angulins (which include Angulin-1/LSR, Angulin-2/ILDR1, and Angulin-3/ILDR2) are single-transmembrane proteins having one immunoglobulin-like domain in the extracellular region and one PDZ-binding motif at the carboxy-terminus. Angulins establish tricellular TJs and regulate paracellular barrier functions.

TJs form barrier layers in various parts of the body, and their disruption is implicated in the pathology of various diseases. For example, an intact intestinal barrier is crucial for immune homeostasis and its impairment activates the immune system and may result in chronic inflammation. The epithelial cells of the intestinal barrier are connected by tight junctions, which form an anastomosing network sealing adjacent epithelial cells. Each individual component of the tight junction network closely interacts with each other to form an efficient intestinal barrier.

The degradation of barrier layer integrity, including the disruption of tight junctions, can result in various diseases. For example, the degradation of the blood-brain barrier (BBB) can lead to the entry of harmful substances, such as pathogens, toxins, and inflammatory cells, into the brain, which can trigger an inflammatory response and lead to neurological disorders such as multiple sclerosis, Alzheimer's disease, Parkinson's disease, and stroke. Similarly, the degradation of the gut barrier, also known as the intestinal barrier, can result in the entry of harmful substances, such as bacteria, bacterial virulence factors such as the gingipains produced by the organism P. gingivalis or candidalysins produced by C. albicans, or other toxins, into the bloodstream, leading to inflammation and immune activation or dysregulation. This can contribute to the development of various gastrointestinal diseases, such as inflammatory bowel disease, celiac disease, Crohn's disease, leaky gut syndrome, and irritable bowel syndrome. The degradation of the skin barrier can result in the entry of environmental toxins, allergens, and pathogens, leading to inflammation and skin disorders such as eczema, psoriasis, and acne. The degradation of the lung barrier, which includes the airway epithelium, basement membrane, and capillary endothelium, can result in the entry of environmental pollutants and allergens, leading to inflammation and respiratory diseases such as asthma, chronic obstructive pulmonary disease (COPD), and lung cancer.

In the case of the BBB, degradation of this barrier layer can result in neuroinflammation through several mechanisms. Firstly, the BBB plays a crucial role in maintaining the homeostasis of the brain microenvironment by regulating the entry of molecules and cells from the bloodstream into the brain. The breakdown of the BBB due to the degradation of the barrier layer integrity may result in the entry of harmful substances, such as pathogens, their virulence factors, toxins, and inflammatory white blood cells, into the brain. This can trigger an inflammatory response, leading to the release of pro-inflammatory cytokines, chemokines, and other mediators, which activate resident immune cells, such as microglia and astrocytes, to release additional inflammatory molecules and perpetuate the inflammatory response. Secondly, the BBB is also important for preventing the entry of immune cells into the brain. In the presence of a compromised BBB, immune cells, such as T cells, B cells, and monocytes, may enter the brain and trigger an immune response, leading to the release of inflammatory molecules and the activation of resident immune cells. Thirdly, the BBB also plays a role in maintaining the balance of neurotransmitters and other signaling molecules in the brain. The disruption of the BBB may result in an imbalance of neurotransmitters, leading to altered neuronal function and increased susceptibility to neuroinflammation. Finally, the BBB is also important for maintaining the integrity of the extracellular matrix and the basement membrane in the brain, which provide structural support to brain cells. The degradation of these structures due to the breakdown of the BBB may result in the activation of pro-inflammatory signaling pathways, leading to neuroinflammation.

Neuroinflammation plays a major role in neurodegenerative diseases and is often a secondary reaction to earlier brain injury. Such brain injury may include or arise from, for example, brain trauma, brain cancer, or the presence within the brain of amyloid beta (Aβ) or hyperphosphorylated tau. Activation of microglia by these insults may elicit the expression of pro-inflammatory cytokines such as interleukin (IL)-1β, IL-6, and tumor necrosis factor-α (TNF-α). These cytokines, in turn, stimulate inducible nitric oxide synthase (iNOS) and NO production with abnormal phagocytic activity. These phages may contribute to the neuronal degeneration which is characteristic of the pathogenesis of various neurodegenerative diseases such as, for example, Alzheimer's disease (AD), Parkinson's disease (PD), Huntington's disease (HD), multiple sclerosis (MS) and amyotrophic lateral sclerosis (ALS).

As previously noted, certain polyphenols may be utilized in the compositions, systems and methodologies disclosed herein to reduce inflammation, including neuroinflammation. Neuroinflammation may be suppressed, and neuronal death may be avoided, through the use of suitable anti-inflammatory agents such as, for example, polyphenols.

One specific example of a polyphenol which suppresses inflammation is quercetin (see FIG. 3). Quercetin has been found to block the inflammation induced by certain toxic insults by repressing the overproduction of NO, iNOS enzyme, and other inflammatory genes. Quercetin is also found to inhibit lipopolysaccharide (LPS)/Interferon γ induced inflammation. Quercetin also decreases the expression of pro-inflammatory cytokines (e.g., TNF-α and IL-1α) in astrocytes, diminishes microglial activated neuronal cell death, and exhibits potent anti-neuroinflammatory activity by repressing TNF-α via amplifying the NFkB signaling pathway.

Similarly, curcumin (a compound found in turmeric) has been shown to have potent anti-inflammatory effects by inhibiting the activity of several inflammatory pathways in the body, including NF-kappaB and COX-2. It has also been shown to modulate the expression of inflammatory cytokines such as TNF-alpha, interleukin-1 beta (IL-1p), and interleukin-6 (IL-6). As a result, curcumin has potential therapeutic effects in the treatment a variety of inflammatory diseases, including arthritis, ulcerative colitis, and chronic obstructive pulmonary disease (COPD).

Another example of a polyphenol that has been shown to suppress inflammation is resveratrol, which is found in grapes, berries, and red wine. Resveratrol has been found to have anti-inflammatory effects by inhibiting the activity of inflammatory enzymes such as COX-2 and iNOS, and by modulating the expression of inflammatory cytokines such as TNF-alpha and IL-6. In addition, resveratrol has been found to have antioxidant properties, which can help to reduce oxidative stress and inflammation in the body. As a result, resveratrol has potential therapeutic effects in the treatment a variety of inflammatory diseases, including arthritis, asthma, and cardiovascular disease.

Yet another example of a polyphenol that has been shown to suppress inflammation is epigallocatechin gallate (EGCG), which is found in green tea. EGCG has been found to have anti-inflammatory effects by inhibiting the activity of inflammatory enzymes such as COX-2 and iNOS, and by modulating the expression of inflammatory cytokines such as TNF-alpha and IL-1β. In addition, EGCG has been found to have antioxidant properties, which can help to reduce oxidative stress and inflammation in the body. As a result, EGCG has potential therapeutic effects in the treatment of a variety of inflammatory diseases, including rheumatoid arthritis, inflammatory bowel disease, and neurodegenerative diseases.

Still another example of a polyphenol that has been shown to suppress inflammation is oleocanthal, which is found in extra-virgin olive oil. Oleocanthal has been found to have anti-inflammatory effects by inhibiting the activity of COX-1 and COX-2 enzymes, similar to the way that non-steroidal anti-inflammatory drugs (NSAIDs) work. However, unlike NSAIDs, which can have negative side effects, oleocanthal has been shown to have a much milder effect on the stomach lining, making it a potentially safer alternative for managing inflammation. In addition to its anti-inflammatory properties, oleocanthal has also been found to have antioxidant and neuroprotective properties. As a result, oleocanthal has potential therapeutic effects in the treatment of a variety of inflammatory diseases, including arthritis, cancer, and neurodegenerative diseases.

Another example of a polyphenol that has been shown to suppress inflammation is gingerol, which is found in ginger. Gingerol has been found to have anti-inflammatory effects by inhibiting the production of inflammatory cytokines such as TNF-alpha and IL-1, and by suppressing the activity of enzymes such as COX-2 and iNOS. Gingerol has also been found to have antioxidant properties, which can help to reduce oxidative stress and inflammation in the body. As a result, gingerol has potential therapeutic effects in a variety of inflammatory diseases, including arthritis, ulcerative colitis, and cardiovascular disease.

Various polyphenols may be utilized in the compositions and methodologies disclosed herein. These include, for example, quercetin, curcumin, ellagic acid, epigallocatechin gallate (EGCG), raspberry ellagitannin, theaflavin-3-gallate, tea phenols, the phenol-saccharide conjugate puerarin, the synthetic ellagitannin known as tellimagrandin II, and the various polyphenols and related compounds disclosed in TABLES 1-2 below. The use of quercetin is preferred in that it limits inflammation through the suppression of some inflammatory cytokines, while promoting a salutary symbiotic microbiota in the oral cavity, the gut and possibly other areas of the body. Thus, the use of quercetin in the compositions described herein may make them especially suitable for treating or preventing dysbiosis of the type commonly associated with periodontal disease.

However, it is to be noted that polyphenols other than quercetin may have beneficial effects on the microbiome. Proanthocyanidins (PACs), which are found in many fruits (including cranberries, blueberries, and grapes), are one example. PACs have been found to have prebiotic effects, which means they can selectively stimulate the growth and activity of beneficial bacteria in the gut, such as Bifidobacteria and Lactobacilli. In addition, PACs have been found to have antimicrobial properties, which can help to reduce the growth of harmful bacteria in the gut. Consequently, PACs may have potential therapeutic effects in a variety of conditions related to the gut microbiome, including irritable bowel syndrome (IBS), inflammatory bowel disease (IBD), and colorectal cancer.

Ellagitannins are another example of a polyphenol that is beneficial to the microbiome. These polyphenols are found in many fruits, including pomegranates, strawberries, and raspberries. Ellagitannins are converted to ellagic acid by gut bacteria, which has been shown to have prebiotic effects, selectively promoting the growth of beneficial bacteria such as Bifidobacteria and Lactobacilli. In addition, ellagitannins have been found to have anti-inflammatory and antioxidant properties, which can help to reduce oxidative stress and inflammation in the gut. As a result, ellagitannins may have potential therapeutic effects in a variety of conditions related to the gut microbiome, including colorectal cancer, inflammatory bowel disease (IBD), and metabolic disorders.

Another example of a polyphenol that is beneficial to the microbiome is resveratrol, which is found in grapes, red wine, and peanuts. Resveratrol has been found to have prebiotic effects by selectively promoting the growth of beneficial bacteria in the gut, such as Bifidobacteria and Lactobacilli. In addition, resveratrol has been found to have anti-inflammatory and antioxidant properties, which can help to reduce inflammation and oxidative stress in the gut. Resveratrol has also been found to have protective effects against colon cancer by modulating the gut microbiome. As a result, resveratrol has been studied for its potential therapeutic effects in a variety of conditions related to the gut microbiome, including obesity, type 2 diabetes, and inflammatory bowel disease (IBD).

Catechins are another example of a polyphenol that is beneficial to the microbiome. Catechins are flavonoids which are found in tea, especially green tea. Catechins have been found to have prebiotic effects by selectively promoting the growth of beneficial bacteria in the gut, such as Bifidobacteria and Lactobacilli. In addition, catechins have been found to have antimicrobial properties, which can help to reduce the growth of harmful bacteria in the gut. Catechins have also been found to have anti-inflammatory and antioxidant properties, which can help to reduce inflammation and oxidative stress in the gut. As a result, catechins may have potential therapeutic effects in a variety of conditions related to the gut microbiome, including obesity, type 2 diabetes, and inflammatory bowel disease (IBD).

Without wishing to be bound by theory, the various polyphenols disclosed herein may reduce inflammation by a variety of pathways or mechanisms, some of which have been described above. For example, curcumin (a polyphenol found in turmeric) is believed to operate by inhibiting the activity of pro-inflammatory cytokines and enzymes. Quercetin (a polyphenol found in many fruits and vegetables, including onions, apples, and berries) is believed to operate by inhibiting the activity of pro-inflammatory cytokines and enzymes, and by reducing the production of reactive oxygen species (ROS). Epigallocatechin gallate (EGCG) (a polyphenol found in green tea) is believed to operate by inhibiting the activity of NF-kB and reducing the production of pro-inflammatory cytokines. Anthocyanins (a type of polyphenol found in many fruits and vegetables, including blueberries, blackberries, and cherries) are believed to operate by reducing the production of pro-inflammatory cytokines and oxidative stress.

In some embodiments, the compositions disclosed herein may feature one or more polyphenols and one or more phospholipids. Suitable phospholipids may include diacylglycerides such as, for example, phosphatidic acid (phosphatidate), phosphatidylethanolamine (cephalin), phosphatidylcholine (lecithin), phosphatidylserine, phosphoinositides (including, but not limited to, phosphatidylinositol, phosphatidylinositol phosphate, phosphatidylinositol bisphosphate and phosphatidylinositol trisphosphate), and phosphosphingolipids (including, but not limited to, ceramide phosphorylcholine (sphingomyelin), ceramide phosphorylethanolamine (sphingomyelin) and ceramide phosphoryllipid. The use of blends of blends of quercetin and at least one phospholipid are preferred, and the use of blends of quercetin with lecithin, such as the blend sold by Indena S.p.A. (Lombardy, Italy) under the tradename quercetin phytosome, are especially preferred.

Various biomarkers of barrier layer permeability or integrity may be utilized in the systems and methodologies described herein. These include, without limitation, the protein zonulin; tight junction proteins, such as claudin, occludin, zonula occludens, and junctional adhesion molecules (JAMs); cytokines such as, for example, interleukin-1 (IL-1), interleukin-6 (IL-6), tumor necrosis factor-alpha (TNF-α), IFN-γ, and IL-17A; lipopolysaccharide (LPS); C-type lectin-like receptor 2 (CLEC-2); sugars such as lactulose and mannitol; secretory immunoglobulin A (sIgA); hyaluronic acid (HA); Chitinase-3-like protein 1 (YKL-40); fatty acids; urea and creatinine; surfactant protein D (SP-D); intestinal fatty acid-binding protein (I-FABP); calprotectin; and matrix metalloproteinases such as, for example, MMP-3 and MMP-9. It will be appreciated that the foregoing biomarkers may be used alone or in various combinations.

In various embodiments, the biomarker can be determined using one or more biomolecules, including peptides, nucleic acids, carbohydrates, fatty acids, organelles, cellular bodies, and/or combinations thereof (e.g., a glycoprotein). Peptides can include oligopeptides, peptide fragments, epitopes, full length proteins, enzymes, etc. Nucleic acids may include DNA, RNA, and combinations thereof. RNA molecules can include messenger RNA (mRNA), pre-mRNA, small RNAs (e.g., miRNA, siRNA, etc.). Certain instances measure the biomarker using one or more exosomes.

Zonulin is a protein that regulates the opening and closing of tight junctions between cells in the intestinal barrier. Increased levels of zonulin in the blood are associated with increased intestinal permeability, which is commonly seen in various gastrointestinal disorders. Inflammatory cytokines, such as interleukin-1 (IL-1), interleukin-6 (IL-6), and tumor necrosis factor-alpha (TNF-α), can be released in response to damage to the barrier layer. Increased levels of these cytokines in the blood or tissue can indicate increased permeability or damage to the barrier layer. Tight junction proteins, such as claudin, occludin, zonula occludens, and junctional adhesion molecules (JAMs) are important components of the barrier layer. Changes in the expression or localization of these proteins may indicate damage to the barrier layer, disruption of tight junctions, or increased permeability.

Secretory immunoglobulin A (sIgA) is an antibody that is secreted into the gut lumen and plays a role in protecting the intestinal mucosa from pathogens. Decreased levels of sIgA in the gut lumen may be associated with increased intestinal permeability and damage to the intestinal barrier.

Urea and creatinine are waste products that are normally excreted by the kidneys. Elevated levels of these biomarkers in the blood can indicate damage to the renal barrier and decreased kidney function. Creatinine urinary detection kits are available commercially, for example, from ThermoFisher Scientific (Waltham, MA) under the tradename INVITROGEN® (catalog no. EIACUN), and urea nitrogen colorimetric tests (for the quantification and detection of urea nitrogen in serum, plasma, urine, saliva and tissue culture media samples) are available from the same vendor under the same tradename (catalog no. EIABUN).

Fatty acids are important components of cell membranes and play a role in maintaining membrane fluidity and permeability. Changes in the composition or levels of fatty acids in the blood or tissue can indicate damage to cell membranes and changes in barrier function. Fatty acid tests for essential serum or plasma are available commercially, for example, from Arup Laboratories (Salt Lake City, UT) (catalog no. FA PRO SP).

SP-D is a protein that is produced in the lungs and is important for maintaining lung surfactant and protecting the lungs from infection. Decreased levels of SP-D in the blood or lung fluid may be associated with increased lung permeability and damage to the lung barrier.

I-FABP is a protein that is found in the epithelial cells of the small intestine. Increased levels of I-FABP in the blood may be associated with increased intestinal permeability and damage to the intestinal barrier.

Calprotectin is a protein that is released by immune cells in response to inflammation. Fecal calprotectin levels may be used as a biomarker of intestinal inflammation and damage to the intestinal barrier.

Some biomarkers of barrier layer permeability or integrity that are useful in the systems and methodologies disclosed herein may take then form of physiological measurements. For example, transepithelial electrical resistance (TEER) is a measure of the electrical resistance of a cell layer. The TEER may be used to evaluate the integrity of epithelial cell layers in the skin, gastrointestinal tract, and lung, with a decrease in TEER indicating increased permeability and decreased barrier function. TEER measurement techniques are described, for example, in [Srinivasan B, Kolli A R, Esch M B, Abaci H E, Shuler M L, Hickman J J. TEER measurement techniques for in vitro barrier model systems. J Lab Autom. 2015 April; 20(2):107-26. doi: 10.1177/2211068214561025. Epub 2015 Jan. 13. PMID: 25586998; PMCID: PMC4652793], which is incorporated herein by reference in its entirety.

Similarly, lactulose and mannitol are two sugars that can be used to measure intestinal permeability via the lactulose/mannitol test. This test measures the urinary excretion of lactulose and mannitol after oral administration of these sugars. Lactulose is a large molecule that is normally not absorbed by the intestine, whereas mannitol is a small molecule that is readily absorbed. Increased urinary excretion of lactulose relative to mannitol indicates increased intestinal permeability and damage to the intestinal barrier. The Lactulose/Mannitol urine test is described, for example, in [Musa M A, Kabir M, Hossain M I, Ahmed E, Siddique A, Rashid H, Mahfuz M, Mondal D, Ahmed T, Petri W A, Haque R. Measurement of intestinal permeability using lactulose and mannitol with conventional five hours and shortened two hours urine collection by two different methods: HPAE-PAD and LC-MSMS. PLoS One. 2019 Aug. 8; 14(8):e0220397. doi: 10.1371/journal.pone.0220397. PMID: 31393913; PMCID: PMC6687120], which is incorporated herein by reference in its entirety.

Gastric emptying time is another parameter that may be utilized in the systems and methodologies disclosed herein as a biomarker of intestinal permeability. Gastric emptying time refers to the time it takes for food to move through the stomach and into the small intestine. Delayed gastric emptying has been associated with increased intestinal permeability and gut inflammation. Details of this parameter, and how it may be measured, may be found, for example in [Peter L. Lu, Carlo Di Lorenzo, Chapter 28—Gastric Motility Disorders, Editor(s): Robert Wyllie, Jeffrey S. Hyams, Marsha Kay, Pediatric Gastrointestinal and Liver Disease (Sixth Edition), Elsevier, 2021, Pages 293-302.e3, ISBN 9780323672931, https://doi.org/10.1016/B978-0-323-67293-1.00028-1], which is incorporated herein by reference in its entirety.

The urinary albumin-to-creatinine ratio (UACR) is another parameter that may be utilized in the systems and methodologies disclosed herein. UACR is a measure of the amount of albumin (a protein) relative to creatinine (a waste product) in the urine. Increased UACR levels may be associated with increased permeability of the renal barrier and damage to the kidneys. Albumin and creatinine blood tests are described for example, at MedlinePlus.gov at [https://medlineplus.gov/ency/article/003480.htm] and [https://medlineplus.gov/ency/article/003475.htm], respectively, both of which are incorporated herein by reference in their entirety.

Various testing methodologies for biomarkers may be utilized in the systems and methodologies disclosed herein. These include, for example, immunoassays such as ELISA (Enzyme-Linked Immunosorbent Assay) kits or reagents, Western blotting, quantitative PCR (qPCR) and its variations, sequencing (e.g., next-generation sequencing), a biosensor such sequencing can sequence DNA, RNA, and/or other nucleic acids. In such a kit, the antigen (which may be, for example, the protein zonulin) is typically immobilized on a substrate and then complexed with an antibody linked to a reporter enzyme. Detection of the antigen may then be accomplished by measuring the activity of the reporter enzyme after incubation with the appropriate substrate to produce some measurable product.

ELISA kits for zonulin are commercially available, for example, from Elabscience (Houston, TX) (catalog no. E-EL-H5560) and Eagle Biosciences (Amherst, NH) (catalog no. KR5601). ELISA kits for IFN-γ are commercially available, for example, from Proteintech (Rosemont, IL) under the tradename AuthentiKine™ (catalog no. KE00146). ELISA kits for IL17A are commercially available, for example, from RayBiotech (Peachtree Corners, GA) (catalog no. ELH-IL17-1). ELISA kits for LPS are commercially available, for example, from MyBiosource.com (San Diego, CA) (catalog no. MBS702450). ELISA kits for CLEC-2 are commercially available, for example, from RayBiotech (Peachtree Corners, GA) (catalog no.ELH-CLEC2-1). ELISA kits for MMP-9 are commercially available, for example, from Proteintech (Rosemont, IL) (catalog no. KE00164). ELISA kits for MMP-3 are commercially available, for example, from RayBiotech (Peachtree Corners, GA) under the tradename IQELISA™ (catalog no. IQH-MMP3-1). ELISA kits for albumin are commercially available, for example, from ThermoFisher Scientific (Waltham, MA) under the tradename INVITROGEN® (catalog no. EHALB). ELISA kits for creatinine are commercially available, for example, from RayBiotech (Peachtree Corners, GA) (catalog no. MA-CTN-2). ELISA kits for Secretory IgA are commercially available, for example, from Eagle Biosciences (Amherst, NH) (catalog no. SGA35-KO1). ELISA kits for calprotectin are commercially available, for example, from Biotechne R&D Systems (Minneapolis, MN) (catalog no. DSFPDO). ELISA kits for IFABP/FABP2 are commercially available, for example, from Elabscience (Houston, TX) (catalog no. E-EL-H0159). ELISA kits for albumin are commercially available, for example, from Abcam Plc (Boston, MA) under the tradename SimpleStep® (catalog no. ab239431).

In some embodiments of the systems and methodologies disclosed herein, magnetic resonance imaging (MRI), preferably dynamic contrast-enhanced MRI (DCE-MRI), and even more preferably DCE-MRI, may be used in conjunction with post-processing analysis with improved spatial and temporal resolutions, may be utilized to quantify a BBB regional permeability (Kₜᵣₐₙₛ) constant in a subject. Kₜᵣₐₙₛ may be used as a metric to assess BBB integrity in a subject, which may inform a treatment regimen for the subject. In some cases, the integrity of the BBB in the hippocampus (and especially in the CA1 and DG regions thereof) may be of particular interest. In this respect, it is to be noted that the BBB is not monolithic but can vary significantly and can depend, for example, on local capillary density and glial cell physiology. Moreover, the tight junction morphology and permeability of the BBB at any location is at least partially controlled by claudins, a family of transmembrane proteins. Thus, for example, claudin-1, claudin-2 and claudin-5 have been associated, respectively, with linear, ruffled and spiked tight junction morphologies. Details of the foregoing methodology may be found, for example, in [Bae J, Zhang J, Wadghiri Y Z, Minhas A S, Poptani H, Ge Y, Kim S G. Measurement of blood-brain barrier permeability using dynamic contrast-enhanced magnetic resonance imaging with reduced scan time. Magn Reson Med. 2018 October; 80(4):1686-1696. doi: 10.1002/mrm.27145. Epub 2018 Mar. 5. PMID: 29508443; PMCID: PMC6340058], which is incorporated herein by reference in its entirety.

In some embodiments of the systems and methodologies disclosed herein, the cerebrospinal fluid (CSF)/plasma albumin quotient (Q_{Alb}) may also be used as a biomarker of BBB integrity. In particular, it has been found that, in some applications, correlations exist between Q_{Alb} and Kₜᵣₐₙₛ. Similarly, FITC-albumin microangiography may be utilized as a useful tool in assessing BBB integrity, due to its ability to provide indications of changes in BBB functionality due to its ability to provide simultaneous assessment of vascular architecture and permeability to serum proteins. Details of the foregoing methodology may be found, for example, in [Altered CSF Albumin Quotient Links Peripheral Inflammation and Brain Damage in MS, Marco Puthenparampil, Paula Tomas-Ojer, Thorsten Hornemann, Andreas Lutterotti, Ilijas Jelcic, Mario Ziegler, Andreas J. Hulsmeier, Carolina Cruciani, Wolfgang Faigle, Roland Martin, Mireia Sospedra, Neurol Neuroimmunol Neuroinflamm March 2021, 8 (2) e951; DOI: 10.1212/NXI.0000000000000951], which is incorporated herein by reference in its entirety.

In some embodiments of the systems and methodologies described herein, levels of biomarkers (such as, for example, immunoglobulins or metabolites) in bodily fluids or samples may be correlated with, and used to quantify or qualify, oral or gut dysbiosis or the dysbiosis of other microbiota.

For example, secretory IgA (sIgA) is an immunoglobulin that is present in high levels in the gut and plays a key role in protecting against pathogens. Decreased levels of sIgA in the gut may be associated with dysbiosis and increased susceptibility to infections. Immunoglobulin M (IgM) is the first antibody produced in response to an infection and plays a key role in the immune response. Decreased levels of IgM in the gut may be associated with dysbiosis and increased susceptibility to infections. Immunoglobulin G (IgG) is an immunoglobulin that is produced in response to both acute and chronic infections. Increased levels of IgG in the gut may be associated with dysbiosis and chronic inflammation.

Since saliva is a readily available bio-sample, it may be useful to establish correlations between the levels of the foregoing (or other) immunoglobulins in saliva and the levels of these immunoglobulins in the gut, since correlations between these two would in turn allow clinical practitioners to establish correlations between the levels of these immunoglobulins in saliva and gut dysbiosis.

The levels of immunoglobulins such as sIgA, IgM, and IgG in saliva can potentially be correlated with the levels of these immunoglobulins in the gut through various means. For example, fecal immunoglobulin analysis can be used to assess the levels of immunoglobulins such as sIgA, IgM, and IgG in the gut. By comparing the levels of these immunoglobulins in fecal samples with those in saliva samples from the same individual, clinical practitioners can potentially establish a correlation between the two.

Similarly, serum immunoglobulin analysis may be utilized to assess the levels of immunoglobulins in the bloodstream. Since some of these immunoglobulins are produced in response to gut microbes, changes in their levels may reflect changes in the gut microbiome. By comparing the levels of these immunoglobulins in serum samples with those in saliva samples from the same individual, clinical practitioners can potentially establish a correlation between the two.

In an analogous manner, microbial sequencing of saliva samples can provide information about the composition of the oral microbiome, which may be correlated with the gut microbiome. By comparing the microbial composition of saliva samples with fecal samples from the same individual, clinical practitioners can potentially establish a correlation between the two.

The levels of various metabolites in bodily fluids or samples may be correlated with, and used to quantify or qualify, oral or gut dysbiosis or the dysbiosis of other microbiota. These include, without limitation, short-chain fatty acids (SCFAs), indole metabolites, lipid metabolites, amino acid metabolites, trimethylamine (TMA) and trimethylamine-N-oxide (TMAO), bile acids, polyamines, neurotransmitters, phenolic compounds, uremic toxins, folate, and tryptamine.

SCFAs are produced by gut bacteria during the fermentation of dietary fibers. They play an important role in maintaining gut health by promoting the growth of beneficial bacteria, modulating immune responses, and regulating intestinal motility. Reduced levels of SCFAs may be associated with gut dysbiosis and various gut-related disorders.

Indole metabolites are produced by gut bacteria from the breakdown of dietary tryptophan. They have been implicated in various physiological processes such as immune modulation, gut motility, and neuroprotection. Changes in the levels of indole metabolites have been observed in individuals with gut dysbiosis and have been associated with inflammatory bowel disease and colorectal cancer.

Lipid metabolites such as phosphatidylcholine and sphingomyelin have been found to be altered in individuals with gut dysbiosis. These changes have been linked to the development of metabolic disorders such as obesity, insulin resistance, and non-alcoholic fatty liver disease.

Amino acid metabolites such as tyrosine, phenylalanine, and tryptophan have been found to be associated with gut dysbiosis. Changes in the levels of these metabolites have been linked to various neurological and psychiatric disorders such as depression, anxiety, and autism spectrum disorder.

TMA and TMAO are produced by gut bacteria during the metabolism of choline and L-carnitine. Elevated levels of TMAO have been associated with an increased risk of cardiovascular disease, and gut dysbiosis has been linked to an increase in TMAO levels.

Bile acids are produced by the liver and are involved in the digestion and absorption of dietary fats. They also have important signaling functions in the gut. Alterations in the gut microbiota can result in changes in the composition of bile acids, which has been linked to the development of various gut-related disorders such as inflammatory bowel disease and colon cancer.

Polyamines such as putrescine, spermidine, and spermine are important for gut health and are involved in various physiological processes such as cell growth and differentiation. Changes in the levels of polyamines have been observed in individuals with gut dysbiosis and have been associated with the development of gut-related disorders such as colorectal cancer.

Neurotransmitters such as serotonin and dopamine are produced by gut bacteria and play an important role in regulating mood, appetite, and behavior. Alterations in the gut microbiota can result in changes in the production of neurotransmitters, which has been linked to various neurological and psychiatric disorders.

Phenolic compounds such as phenylacetic acid, p-cresol, and indole-3-acetic acid are produced by gut bacteria during the metabolism of aromatic amino acids. Elevated levels of these compounds have been associated with gut dysbiosis and have been linked to the development of various gut-related disorders such as inflammatory bowel disease and colorectal cancer.

Uremic toxins such as indoxyl sulfate and p-cresyl sulfate are produced by gut bacteria from the metabolism of dietary proteins. These toxins are normally excreted by the kidneys, but in individuals with impaired renal function, they can accumulate in the body and contribute to the development of various metabolic and cardiovascular disorders.

Folate is an essential vitamin that is produced by gut bacteria and is involved in various physiological processes such as DNA synthesis and repair. Alterations in the gut microbiota can result in changes in the production of folate, which has been linked to various gut-related disorders such as inflammatory bowel disease and colon cancer.

Tryptamine is a neurotransmitter that is produced by gut bacteria from the metabolism of tryptophan. Changes in the levels of tryptamine have been observed in individuals with gut dysbiosis and have been associated with various neurological and psychiatric disorders such as depression and anxiety.

Various analytical techniques may be utilized to quantify biomarker levels in bodily fluids. These include, for example, gas chromatography-mass spectrometry (GC-MS) and liquid chromatography-mass spectrometry (LC-MS). These techniques can be utilized to accurately measure the levels of biomarkers in bodily fluids, thus providing valuable information on the extent of gut dysbiosis.

Of course, it will be appreciated that the foregoing processes may be automated and that any statistical correlations performed therein may be based on statistical characterizations of various populations. These include, without limitation, the determination of mean and variance, the determination of correlation coefficients (such as, for example, the Pearson correlation coefficient), the use of statistical methodologies to determine if there is a significant difference between the means of two groups of data (including, for example, the Student's t-test), and the like.

In some embodiments of the systems and methodologies disclosed herein, multivariate analysis can be used to assess dysbiosis in a subject based on samples taken from distinct microbiomes in the subject by analyzing multiple variables simultaneously, including the relative abundance of different microbial taxa in each microbiome, as well as any clinical or demographic variables that may affect the microbiome composition. This type of analysis can help identify patterns and relationships between different microbiomes and determine the degree of dysbiosis in each microbiome, as well as any correlations between dysbiosis in different microbiomes.

For example, principal component analysis (PCA) can be used to visualize the variation between different microbiomes and identify potential clustering of samples based on their microbial composition. Canonical correlation analysis (CCA) can be used to identify correlations between different microbiomes and any associated clinical or demographic variables. Other multivariate techniques, such as discriminant analysis or machine learning algorithms, can be used to identify biomarkers or predictive models for dysbiosis in different microbiomes. Overall, multivariate analysis can provide a comprehensive and integrated assessment of dysbiosis in a subject based on samples taken from distinct microbiomes.

Other statistical or mathematical techniques may also be utilized to assess dysbiosis in a subject based on samples taken from distinct microbiomes in the subject. Some of these techniques include alpha and beta diversity measures, network analysis, correlation analysis, machine learning algorithms, and time series analysis. For example, alpha diversity may be utilized to measure the diversity within each microbiome, while beta diversity may be utilized to measure the diversity between different microbiomes. These measures may help to identify differences in microbiome composition and diversity between healthy and dysbiotic subjects. Network analysis may be utilized to identify interactions between different microbial taxa and their co-occurrence patterns within and between different microbiomes. This may help identify potential dysbiotic networks that are associated with disease. Correlation analysis may be used to identify correlations between different microbial taxa and between different microbiomes. This may help identify potential dysbiotic associations that are associated with disease. Machine learning algorithms may be utilized to identify dysbiotic biomarkers or predictive models for disease based on the composition of different microbiomes. Time series analysis may be utilized to analyze changes in microbiome composition over time and to identify potential dysbiotic trends that are associated with disease. One skilled in the art will appreciate that the particular choice of statistical or mathematical techniques will depend on the questions to be answered and the available data, it being understood that multiple techniques may need to be used in combination to provide a comprehensive assessment of dysbiosis in a subject based on samples taken from distinct microbiomes.

Concentrations or levels of biomarkers may be ascertained in various media or biological samples in the systems and methodologies disclosed herein. Preferably, concentrations of the biomarkers disclosed herein will be ascertained in whole blood, blood serum, blood plasma, cerebral spinal fluid (CSF), saliva, sputum, subgingival fluids, and other biological materials.

Various reference ranges or values (which may be scalar or vectoral) may be established for the biomarkers disclosed herein. Preferably, suitable statistical methods may be utilized to determine whether any deviation in the measured values of these biomarkers from the reference values are statistically significant. For example, in some applications, a standard deviation may be considered statistically significant.

In some embodiments of the compositions, systems and methodologies disclosed herein, it may be advantageous to suppress pathogenic virulence factors as part of a treatment. For example, the endogenous oral pathogen P. gingivalis (Pg) can deactivate LL-37 by secreting protease enzymes called gingipains, which cleave the peptide bond between two specific amino acids (phenylalanine and arginine) in the LL-37 molecule. This cleavage disrupts the structure of LL-37 and reduces its antimicrobial activity and impairs its important immunomodulatory activities. Hence, suppression of such virulence factors in conjunction with the expression of LL-37 may provide a more desirable immunological response.

Pg dampens all aspects of interferon (IFN) signaling in a manner that is strikingly similar to the IFN suppression employed by multiple viral pathogens. Pg suppresses IFN production by down-regulating several IFN regulatory factors (IRFs 1, 3, 7, and 9), proteolytically degrading STAT1 and suppressing the nuclear translocation of the ISGF3 complex, resulting in profound and systemic repression of multiple interferon-stimulated genes. Pg-induced IFN paralysis is observed not only in murine models but was also in the oral tissues of human periodontal disease patients, where overabundance of Pg has been correlated with suppressed TFN generation. Mechanistically, multiple virulence factors and secreted proteases produced by Pg transcriptionally suppress IFN promoters and also cleave IFN receptors, making cells refractory to exogenous IFN and inducing a state of broad IFN paralysis. Thus, Pg behaves with an equivalence to viruses in the down-regulation of host IFN signaling. [Rodriguez-Hernandez C J, Sokoloski K J, Stocke K S, Dukka H, Jin S, Metzler M A, Zaitsev K, Shpak B, Shen D, Miller D P, Artyomov M N, Lamont R J, Bagaitkar J. Microbiome-mediated incapacitation of interferon lambda production in the oral mucosa. Proc Natl Acad Sci USA. 2021 Dec. 21; 118(51):e2105170118. doi: 10.1073/pnas.2105170118. Erratum in: Proc Natl Acad Sci USA. 2022 Aug. 30; 119(35):e2212111119. PMID: 34921113; PMCID: PMC8713781; the disclosure of which is hereby incorporated by reference in its entirety].

Without wishing to be bound by theory, it is believed that some polyphenols may be able to suppress the ability of Pg to dampen IFN signaling. This may occur by virtue of the ability of some polyphenols to modulate the activity of certain signaling pathways that are involved in innate immune responses. More specifically, NF-kB is a transcription factor that plays a key role in regulating the expression of many pro-inflammatory cytokines and chemokines. While NF-kB is important for the activation of the innate immune response and the clearance of bacterial pathogens, its excessive or prolonged activation can lead to chronic inflammation and tissue damage. In addition, activation of the NF-kB pathway has been shown to dampen the activity of interferon (IFN) signaling, which is an important part of the innate immune response to viral and bacterial infections.

It has been found that some polyphenols can inhibit the activity of NF-kB, a transcription factor that plays a key role in regulating the expression of many pro-inflammatory cytokines and chemokines. By inhibiting NF-kB activity, polyphenols may be able to reduce the production of pro-inflammatory molecules that are induced by Pg and may help to restore the IFN signaling pathway.

For example, curcumin has been found to inhibit NF-kB activation by inhibiting the phosphorylation of the inhibitor of kappa B (IκB) protein, which prevents its degradation and subsequent release of NF-kB. Resveratrol has been found to inhibit NF-kB activation by suppressing the activity of the IκB kinase (IKK) complex, which is responsible for the phosphorylation and degradation of IκB. Epigallocatechin-3-gallate (EGCG) has been found to inhibit NF-kB activation by blocking the phosphorylation and degradation of IκB. Quercetin has been found to inhibit NF-kB activation by blocking the nuclear translocation of NF-kB and suppressing the activity of the IKK complex.

By inhibiting NF-kB activity, it may be possible to promote or protect the IFN signaling pathway. This is because NF-kB has been shown to inhibit the activity of several key components of the IFN signaling pathway, including interferon regulatory factor (IRF) proteins and signal transducer and activator of transcription (STAT) proteins. By reducing the activity of NF-kB, it may be possible to allow these components to function more effectively and promote IFN signaling. In addition, it has also been found that inhibiting NF-kB activity may enhance the expression of certain interferon-stimulated genes (ISGs), which are important effectors of the innate immune response. By enhancing the expression of ISGs, it may be possible to promote the activity of the IFN signaling pathway and enhance the clearance of viral and bacterial pathogens.

It has also been found that some polyphenols may be able to modulate the activity of toll-like receptors (TLRs), which are key components of the innate immune response that recognize bacterial pathogens and activate downstream signaling pathways. Pg has been shown to dampen the activity of TLRs, which may contribute to its ability to evade the immune response. Some polyphenols, by contrast, have been found to activate TLRs and enhance the innate immune response to bacterial pathogens. This may help to counteract the effects of Pg and restore the IFN signaling pathway. Thus, for example, resveratrol has been found to activate TLR4, which is a receptor for lipopolysaccharides (LPS) found on the surface of gram-negative bacteria. EGCG has been found to activate TLR4 and TLR5, which is a receptor for flagellin, a protein found in bacterial flagella. Luteolin has been found to activate TLR4, which is involved in the recognition of LPS. Curcumin has been found to activate TLR2, which is involved in the recognition of bacterial lipoproteins and peptidoglycans.

Polyphenols may also counteract the activity of gingipains in the suppression of IFN signaling or their role in the pathology of periodontal disease or dementia. Without wishing to be bound by theory, gingipains are believed to play a key role in the pathogenesis of periodontal disease (and possibly the development of both dementia, and cancer) by promoting bacterial invasion, degradation of host proteins, and immune evasion. Some polyphenols may suppress or inactivate gingipains through a few different mechanisms.

Without wishing to be bound by theory, one mechanism by which suppression or inactivation of gingipains by polyphenols is believed to occur involves direct binding to the enzyme and inhibition of its activity. For example, it has been found that some polyphenols, such as epigallocatechin gallate (EGCG), can bind to gingipains and inhibit their proteolytic activity. This may prevent the degradation of host proteins and help to limit the invasiveness of Pg.

In addition, polyphenols may also suppress gingipain activity by modulating the activity of certain signaling pathways. For example, some polyphenols (such as, for example, EGCG, curcumin, resveratrol and quercetin) have been found to inhibit the activity of matrix metalloproteinases (MMPs), which are host enzymes that can be activated by gingipains and contribute to tissue destruction. By inhibiting MMP activity, polyphenols may help to limit the damage caused by Pg and promote tissue repair.

Another potential mechanism by which polyphenols may suppress gingipain activity is through modulation of bacterial gene expression. It has been found that some polyphenols may influence the expression of genes involved in bacterial virulence and metabolism, including gingipain genes. By downregulating the expression of gingipain genes, polyphenols may reduce the production of gingipains and help to limit their activity. A listing of some polyphenols which may exhibit activity in suppressing gingipains or the expression of gingipain genes is included in TABLE 1. TABLE 2 depicts some members of a family of polyphenols (anthocyanidins) which may exhibit activity in suppressing gingipains.

TABLE 1
Polyphenol Gingipain Inhibitors

| Polyphenol | Structure | Class | Source |
|---|---|---|---|
| Epigallocatechin-3- gallate (EGCG) | 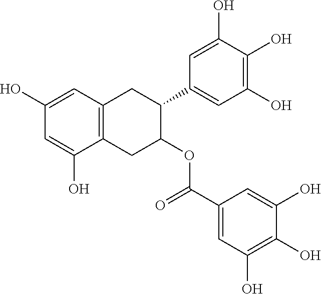 [SMILES: O=C(OC1Cc2c(O)cc(O)cc2C[C@@H]1c1cc(O)c(O)c(O)c1)c1cc(O)c(O)c(O)c1] | Catechin | Green Tea |
| Resveratrol | 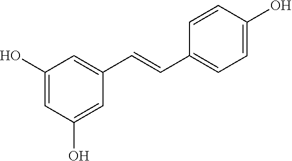 [SMILES: Oc1ccc(/C=C/c2cc(O)cc(O)c2)cc1] | Stilbenoid | Grapes, blueberries, raspberries |
| Curcumin | 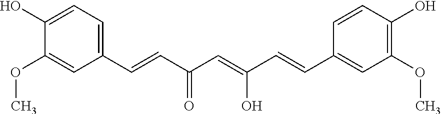 [SMILES: COc1cc(/C=C/C(=O)/C=C(O)/C=C/c2ccc(O)c(OC)c2)ccc1O] | Curcuminoid | Turmeric |
| | Enol Form | | |
| | 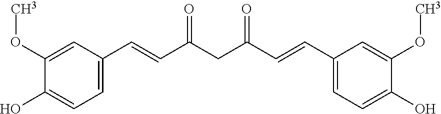 [SMILES: COc1cc(/C=C/C(=O)CC(=O)/C=C/c2ccc(O)c(OC)c2)ccc1O] | | |
| | Keto Form | | |
| Ellagic acid | 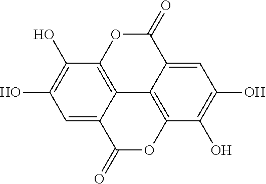 [SMILES: O=c1oc2c(O)c(O)cc3c(=O)oc4c(O)c(O)cc1c4c23] | Pomegranate polyphenols | Pomegranate |
| Punicalagin | 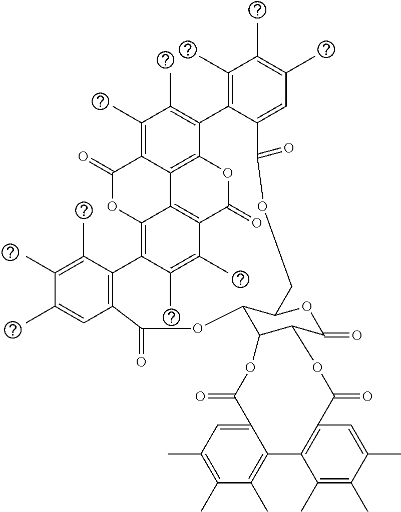 | Ellagitannin | Pomegranate |
| Quercetin | 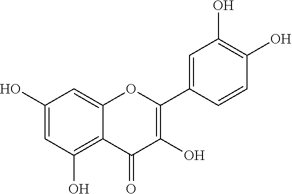 [SMILES: O=c1c(O)c(-c2ccc(O)c(O)c2)oc2cc(O)cc(O)c12] | Flavonoid | Apples, onions, kale |
| Luteolin | 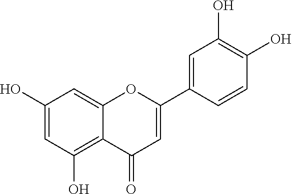 [SMILES: O=c1cc(-c2ccc(O)c(O)c2)oc2cc(O)cc(O)c12] | Flavonoid | Reseda luteola |
| Chlorogenic acid | 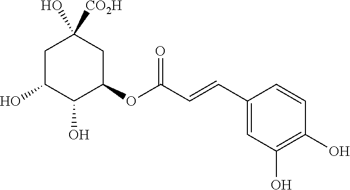 [SMILES: O=C(/C=C/c1ccc(O)c(O)c1)O[C@@H]1C[C@](O)(C(=O)O)C[C@@H](O)[C@H]1O] | Phenolic acid | Coffee, fruits, vegetables |
| Kaempferol | 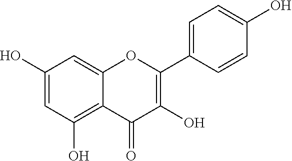 [SMILES: O=c1c(O)c(-c2ccc(O)cc2)oc2cc(O)cc(O)c12] | Flavonoid | Tea, broccoli, grapes |
| Genistein | 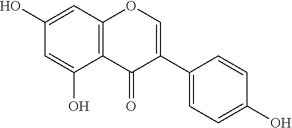 [SMILES: O=c1c(-c2ccc(O)cc2)coc2cc(O)cc(O)c12] | Isoflavone | Soybeans/legumes |
| Naringenin | 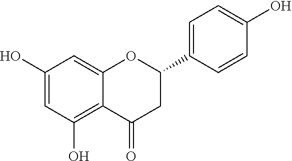 [SMILES: O=C1C[C@@H](c2ccc(O)cc2)Oc2cc(O)cc(O)c21] | Flavonoid | Citrus fruits (e.g., grapefruit, oranges) |
| Fisetin | 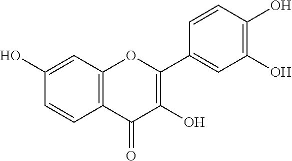 [SMILES: O=c1c(O)c(-c2ccc(O)c(O)c2)oc2cc(O)ccc12] | Flavonoid | Strawberries, apples, onions |
| Myricetin | 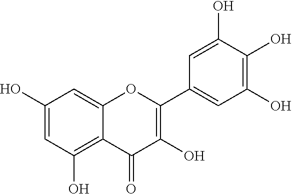 [SMILES: O=c1c(O)c(-c2cc(O)c(O)c(O)c2)oc2cc(O)cc(O)c12] | Flavonoid | Berries, grapes, onions |
| Gallic acid | 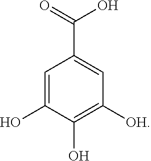 [SMILES: O=C(O)c1cc(O)c(O)c(O)c1] | Phenolic acid | |
| Oleuropein | 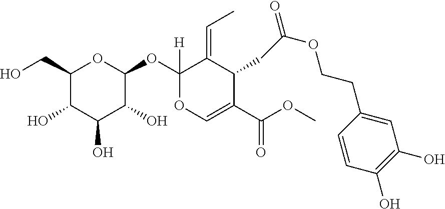 [SMILES: C/C=C1/C(O[C@@H]2O[C@H](CO)[C@@H](O)[C@H](O)[C@H]2O)OC=C(C(=O)OC)[C@H]1CC(=O)OCCc1ccc(O)c(O)c1] | Polyphenol | Olive leaves, olive oil |
| Catechin | 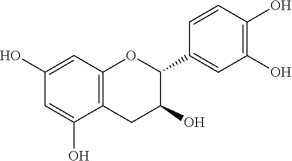 [SMILES: Oc1cc(O)c2c(c1)O[C@H](c1ccc(O)c(O)c1)[C@@H](O)C2] | Flavanol | Green tea, cocoa, some fruits |
| Epicatechin | 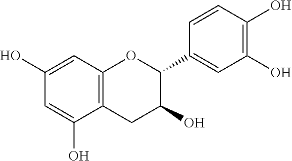 [SMILES: Oc1cc(O)c2c(c1)O[C@H](c1ccc(O)c(O)c1)[C@@H](O)C2] | Flavanol | Tea, cocoa, some fruits |
| Procyanidin | 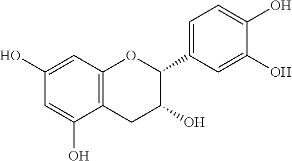 [SMILES: Oc1cc(O)c2c(c1)O[C@H](c1ccc(O)c(O)c1)[C@H](O)C2] | Flavanol | Apples, berries, grapes |
| Cyanidin | 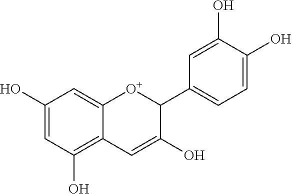 [SMILES: OC1=Cc2c(O)cc(O)cc2[OH+]C1c1ccc(O)c(O)c1] | Flavanol | Apples, berries, grapes |
| Pterostilbene | 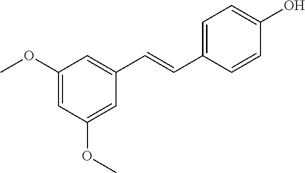 [SMILES: COc1cc(/C=C/c2ccc(O)cc2)cc(OC)c1] | Polyphenol | Blueberries, grapes, peanuts |
| Gingerol | 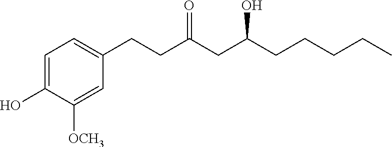 [SMILES: CCCCC[C@H](O)CC(=O)CCc1ccc(O)c(OC)c1] | Polyphenol | Ginger |
| Oleocanthal | 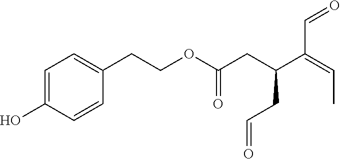 [SMILES: C/C=C(/C=O)[C@@H](CC=O)CC(=O)OCCc1ccc(O)cc1] | Polyphenol | Olive Oil |
| Baicalin | 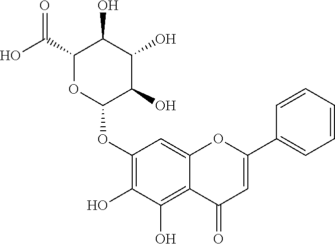 [SMILES: O=C(O)[C@H]1O[C@@H](Oc2cc3oc(-c4ccccc4)cc(=O)c3c(O)c2O)[C@H](O)[C@@H](O)[C@@H]1O] | Flavone glycoside | Roots of Scutellaria baicalensis |
| Anthocyanins | 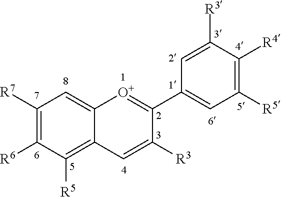 | Flavonoid | Berries, cherries |
| | (see TABLE 2) | | |

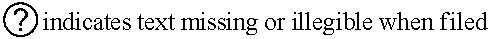

TABLE 2
Selected Anthocyanidins and their Substitutions

| Anthocyanidin | R₃′ | R₄′ | R₅′ | R₃ | R₅ | R₆ | R₇ |
|---|---|---|---|---|---|---|---|
| Aurantinidin | —H | —OH | —H | —OH | —OH | —OH | —OH |
| Cyanidin | —OH | —OH | —H | —OH | —OH | —H | —OH |
| Delphinidin | —OH | —OH | —OH | —OH | —OH | —H | —OH |
| Europinidin | —OCH₃ | —OH | —OH | —OH | —OCH₃ | —H | —OH |
| Pelargonidin | —H | —OH | —H | —OH | —OH | —H | —OH |
| Malvidin | —OCH₃ | —OH | —OCH₃ | —OH | —OH | —H | —OH |
| Peonidin | —OCH₃ | —OH | —H | —OH | —OH | —H | —OH |
| Pctunidin | —OH | —OH | —OCH₃ | —OH | —OH | —H | —OH |
| Rosinidin | —OCH₃ | —OH | —H | —OH | —OH | —H | —OCH₃ |

There is also some evidence to suggest a potential relationship between gingipains and squamous cell carcinomas (SCCs), which are a type of skin cancer. It has been shown that P. gingivalis and its gingipains can promote the growth and invasion of certain cancer cells, including SCCs. In particular, gingipains have been shown to activate a protein called protease-activated receptor-2 (PAR-2), which is involved in the development of SCCs. Additionally, Pg infection has been detected in SCCs of the oral cavity, suggesting a possible link between the bacterium and the development of this type of cancer. See [Inaba H, Sugita H, Kuboniwa M, Iwai S, Hamada M, Noda T, Morisaki I, Lamont R J, Amano A. Porphyromonas gingivalis promotes invasion of oral squamous cell carcinoma through induction of proMMP9 and its activation. Cell Microbiol. 2014 January; 16(1):131-45. doi: 10.1111/cmi.12211. Epub 2013 Sep. 19. PMID: 23991831; PMCID: PMC3939075; the disclosure of which is hereby incorporated by reference in its entirety].

There is also some evidence to suggest a relationship between squamous cell carcinomas (SCCs) and dementia. In particular, some studies have suggested that individuals with a history of cancer, including SCCs, may have a higher risk of developing dementia.

There is also some evidence to suggest a relationship between periodontitis and squamous cell carcinomas (SCCs). Thus, research has shown that individuals with periodontitis may have an increased risk of developing various types of cancer, including SCCs.

In light of the foregoing, the compositions and methodologies disclosed herein may have some utility in treating or preventing SCC, periodontal disease and dementia. Hence, these systems or methodologies utilized as general tools in healthcare or well-being, or as adjuvants to treatments for periodontitis or cancers such as SCC.

One skilled in the art will appreciate that the systems and methodologies disclosed herein, either used alone or as an adjuvant to other treatments or compositions, may be advantageous in addressing a wide variety of diseases, infections and conditions, especially those where the CAMP gene or its proper regulation plays a significant role. These include, for example, various autoimmune diseases, inflammatory conditions, and infections. Some particular, nonlimiting examples of the foregoing include psoriasis, rosacea, inflammatory bowel disease (IBD), cystic fibrosis, atopic dermatitis (eczema), lupus (systemic lupus erythematosus, SLE), rheumatoid arthritis (RA), sepsis, tuberculosis (TB), and periodontitis. The treatment of these conditions with the systems and methodologies disclosed herein illustrate the diverse roles of CAMP gene expression and its encoded peptide LL-37 in modulating immune responses, maintaining barrier integrity, and its potential as a therapeutic target in autoimmune diseases, inflammatory conditions, and infections. In various instances, such treatment can treat a subject. Such a subject can include human and non-human animals. In certain instances, the subject is selected from one or more of a mammal, a primate, an ape, a household pet (e.g., cat, dog, guinea pig, hamster, etc.), a livestock animal (e.g., cow, sheep, goat, pig, etc.), a working animal (e.g., horse, ox, reindeer, etc.). As will be appreciated, certain embodiments combine treatment with a polyphenol composition with an interventional methodology, such as imaging, surgery, etc. Such embodiments can be practices using a cadaver, an anatomical simulant, an anthropomorphic phantom, a virtual simulation, and/or any other acceptable model.

The compositions disclosed herein may be administered as various formulations and by various delivery methods. The selection of a particular composition or route of administration may take into account such factors as the bioavailability of components in the formulation, the need to target specific tissues or cells, or the desire to provide sustained release to maximize therapeutic benefits. In various instances, the composition includes a pharmaceutically acceptable carrier. In addition to oral administration in the form of liquids, tablets or capsules, the compositions described herein may be administered, for example, as nanoformulations, through microencapsulation, as hydrogels, intranasal sprays, transdermal patches, as oral disintegrating films or strips, or as mucoadhesive buccal tablets. For example, polyphenols of the type disclosed herein may be encapsulated in biodegradable polymers such as PLGA (polylactic-co-glycolic acid) to protect them from premature degradation, enhance their absorption in the gastrointestinal tract, and allow for sustained release. Similarly, liposomes and solid lipid nanoparticles may be used to encapsulate polyphenols, improving their solubility and stability, and facilitating targeted delivery to specific tissues, cells, or areas. Additional encapsulation methods that can be used with embodiments include microspheres and nanoparticles.

The compositions disclosed herein may also be combined or used in conjunction with various other therapeutic agents. These may include, for example, antibiotics, probiotics, immunomodulators, antioxidants, anti-inflammatory drugs, cancer chemotherapeutics, antiviral or antimicrobial compositions, other anti-infective agents, anti-tumor agents, anti-cancer agents, vitamin D, and retinoids. The resulting combinations may exhibit enhanced efficacy in upregulating CAMP gene expression or synergistic effects and may offer comprehensive treatment strategies for various diseases. Exemplary antimicrobial agents can include chlorhexidine, cetylpyridinium chloride, and triclosan. In some instances, the compositions disclosed herein may also be combined or used in conjunction with a dietary change or dietary changes.

For example, combining polyphenols with antibiotics may enhance antimicrobial efficacy against resistant strains and reduce the required dose of antibiotics, thereby minimizing side effects. For example, quercetin has been shown to synergize with antibiotics in combating bacterial infections, potentially by mechanisms that impair bacterial cell wall synthesis or DNA replication. See Vipin C, Saptami K, Fida F, Mujeeburahiman M, Rao S S, et al. (2020) Potential synergistic activity of quercetin with antibiotics against multidrug-resistant clinical strains of Pseudomonas aeruginosa. PLOS ONE 15(11): e0241304. https://doi.org/10.1371/journal.pone.0241304; and Stefanovic, O. D. (2018). Synergistic Activity of Antibiotics and Bioactive Plant Extracts: A Study Against Gram-Positive and Gram-Negative Bacteria. InTech. Doi: 10.5772/intechopen.72026.

As a further example, combining polyphenols with probiotics may enhance gut barrier function and immune responses, which may be especially beneficial for conditions such as inflammatory bowel disease (IBD) and irritable bowel syndrome (IBS). For example, polyphenols can synergistically interact with gut microbiota to suppress inflammation and alleviate symptoms of IBD. Polyphenols can also increase the diversity of the gut microbiota, improving the abundance of beneficial bacteria and inhibiting the presence of pathogenic species, thus highlighting their advantages beyond treating IBD. Here, it is to be noted that some polyphenols are not fully absorbed in the small intestine and are metabolized in the colon into compounds with higher anti-inflammatory activity than their precursors. This interaction helps reduce oxidative stress, inhibit secretion of inflammatory cytokines, and protect the gut barrier. See Li, Hao & Christman, Lindsey & Li, Ruiqi & Gu, Liwei. (2020). Synergic Interactions between Polyphenols and Gut Microbiota in Mitigating Inflammatory Bowel Diseases. Food & Function. 11. 10.1039/DOF000713G; see also Calabriso N, Massaro M, Scoditti E, Carluccio MA. Dietary Polyphenols and Their Role in Gut Health. Nutrients. 2023 Jun. 6; 15(12):2650. doi: 10.3390/nu15122650. PMID: 37375554; PMCID: PMC10302038.

In various instances, a probiotic includes a bacterium, bacterial strain, and/or other bacterial composition. In some instances, the bacteria may be selected from a genus, such as Lactobacillus, Bifidobacterium, Streptococcus, and/or any other genus with probiotic activity. In certain instances, the bacteria is selected from one or more species selected from: Lactobacillus acidophilus, Bifidobacterium bifidum, and Streptococcus thermophilus.

For example, combining polyphenols with one or more dietary changes can include changing a diet to a diet that is low in allergenic, inflammatory, and/or pro-inflammatory compounds. In various instances, such diets may include a low FODMAP (fermentable oligosaccharides, disaccharides, monosaccharides and polyols) diet, a gluten-free (or reduced gluten) diet, a low-carbohydrate diet (including, but not limited to, the lion diet, keto diet, carnivore diet, Atkins diet, paleo diet, etc.). In many instances, the dietary change comprises comprise a reduction in consumption of gluten-containing foods.

Various instances can be formulated as a nutraceutical, functional food, and/or other non-pharmacological or non-pharmaceutical formulation. In such instances (e.g., nutraceutical, functional food, etc.) the polyphenol may be formulated with a food-grade carrier.

In various instances, the polyphenol composition is administered in conjunction with another procedure, such as invasive, semi-invasive, minimally invasive, and/or non-invasive procedures such as scaling, root planing, periodontal surgery, a colonoscopy, an endoscopy, open surgery, laparoscopic surgery, tumor resection, tooth cleaning (including tooth brushing), flossing, and/or other procedure.

In many instances, the polyphenol composition is administered until a measured value returns to a target range, a predetermined range, and/or other acceptable value or range of values. Such ranges and values can be determined for an individual based on a number of factors, including (but not limited to) medical history, age, gender, lifestyle factors, and other relevant factors.

Some embodiments of the systems and methodologies described herein may utilize polyphenol phytosomes, that is, complexes where the polyphenol is bound to phospholipids to enhance its absorption and bioavailability. The use of such complexes may increase the effectiveness of the polyphenol in some applications by improving its ability to pass through cell membranes and enter the bloodstream. This may result in potentially better therapeutic effects for some conditions. This is especially true of polyphenols having low solubility under physiological conditions, which may adversely impact their absorption and bioavailability. The use of quercetin phytosome, curcumin phytosome, and green tea phytosome for these purposes is especially preferred. These compositions are commercially available from Thorne Research Inc. (New York, NY)).

For purposes of completeness, various aspects of the present disclosure are set out in the following numbered clauses.
Aspect 1. A method for modulating tight junction (TJ) integrity in a subject, comprising:
   (a) assessing TJ integrity in the subject by quantifying at least one biomarker of TJ integrity in the subject;
   (b) administering to the subject a first composition containing
      (i) at least one polyphenol, and
      (ii) a second substance which is not a polyphenol and which upregulates CAMP gene expression in the subject;
   (c) reassessing TJ integrity in the subject by quantifying at least one biomarker of TJ integrity in the subject; and
   (d) while the value of said at least one biomarker is outside of a target range, repeating steps (b) and (c).
Aspect 2. The method of Aspect 1, wherein assessing TJ integrity in the subject includes quantifying at least one biomarker of TJ integrity in a biological sample obtained from the subject.
Aspect 3. The method of Aspect 2, wherein said biological sample is selected from the group consisting of whole blood, blood plasma, and blood serum.
Aspect 4. The method of Aspect 1, further comprising:
after the value of said at least one biomarker is within a target range, administering the first composition to the subject on a periodic basis.
Aspect 5. The method of Aspect 1, wherein the assessed TJ integrity is of a barrier layer in epithelial cells.
Aspect 6. The method of Aspect 1, wherein the assessed TJ integrity is of a barrier layer in endothelial cells.
Aspect 7. The method of Aspect 1, wherein the assessed TJ integrity is of the boundary between apical and basolateral membrane domains in epithelial and endothelial cells.
Aspect 8. The method of Aspect 1, wherein the assessed TJ integrity is of a barrier layer selected from the group consisting of the blood-brain barrier (BBB) and the intestinal epithelial barrier layer.
Aspect 9. The method of Aspect 1, wherein said at least one polyphenol comprises quercetin.
Aspect 10. The method of Aspect 9, wherein said quercetin is present as a blend with at least one phospholipid.
Aspect 11. The method of Aspect 9, wherein said at least one phospholipid includes lecithin.
Aspect 12. The method of Aspect 1, wherein said at least one polyphenol is a flavonoid.
Aspect 13. The method of Aspect 12, wherein said flavonoid is hydroxyl substituted 3-hydroxy-2-phenylchromen-4-one.
Aspect 14. The method of Aspect 12, wherein said flavonoid is selected from the group consisting of flavonoids and bioflavonoids.
Aspect 15. The method of Aspect 12, wherein said flavonoid is selected from the group consisting of isoflavonoids derived from 3-phenylchromen-4-one (3-phenyl-1,4-benzopyrone) structure.
Aspect 16. The method of Aspect 12, wherein said flavonoid is selected from the group consisting of neoflavonoids, derived from 4-phenylcoumarine (4-phenyl-1,2-benzopyrone) structure.
Aspect 17. The method of Aspect 1, wherein said at least one polyphenol is selected from the group consisting of quercetin, epigallocatechin gallate (EGCG), curcumin, resveratrol, and catechins.
Aspect 18. The method of Aspect 1, wherein the second substance is selected from the group consisting of forskolin, histamine, and butyrate.
Aspect 19. The method of Aspect 1, wherein the TJ integrity biomarker is selected from the group consisting of occludin, claudin-1, zonula occludens-1 (ZO-1), and junctional adhesion molecule A (JAMA).
Aspect 20. The method of Aspect 1, wherein the target range for the TJ integrity biomarker is determined by comparing the biomarker value in the subject to a reference value obtained from a control subject with known TJ integrity.
Aspect 21. The method of Aspect 1, wherein the subject has a condition selected from the group consisting of inflammatory bowel disease, irritable bowel syndrome, leaky gut syndrome, and celiac disease.
Aspect 22. A method for attenuating dysbiosis in a subject, comprising:
   (a) assessing a state of dysbiosis in a subject by quantifying at least one dysbiosis biomarker in the subject;
   (b) administering to the subject a composition containing
      (i) at least one polyphenol, and
      (ii) a second substance which is not a polyphenol and which upregulates CAMP gene expression in the subject; and
   (c) repeating steps (a) and (b) until the value of said at least one dysbiosis biomarker is within a target range.
Aspect 23. The method of Aspect 22, wherein the at least one dysbiosis biomarker is an oral biomarker of dysbiosis.
Aspect 24. The method of Aspect 22, wherein the at least one dysbiosis biomarker is a biomarker of dysbiosis in the oral cavity.
Aspect 25. The method of Aspect 22, wherein the at least one dysbiosis biomarker is a biomarker of dysbiosis in the colon.
Aspect 26. The method of Aspect 22, wherein the at least one dysbiosis biomarker is a biomarker of dysbiosis in the gut.
Aspect 27. The method of Aspect 22, wherein the at least one dysbiosis biomarker includes first and second dysbiosis biomarkers, wherein the first and second dysbiosis biomarkers are biomarkers of dysbiosis in distinct regions of the body, and wherein the regions of the body are selected from the group consisting of the mouth, the gut, and the colon.
Aspect 28. The method of Aspect 22, wherein the at least one dysbiosis biomarker is an index of subgingival microbial dysbiosis.
Aspect 29. The method of Aspect 22, wherein the at least one biomarker is based on the preponderance in a biological sample of at least one periodontitis-associated genera.
Aspect 30. The method of Aspect 29, wherein the periodontitis-associated genera is selected from the group consisting of Fretibacterium, Treponema, Mogibacterium, Peptostreptococcaceae genus 6, and Desulfobulbus.
Aspect 31. The method of Aspect 22, wherein the at least one biomarker is based on the preponderance in a biological sample of at least one health-associated genera.
Aspect 32. The method of Aspect 31, wherein the health-associated genera is selected from the group consisting of Actinomyces and Streptococcus.
Aspect 33. The method of Aspect 22, wherein the dysbiosis biomarker is selected from the group consisting of fecal calprotectin, alpha-1-antitrypsin, zonulin, LPS-binding protein, and beta-defensin 2.
Aspect 34. The method of Aspect 22, wherein the at least one polyphenol is selected from the group consisting of resveratrol, epigallocatechin gallate (EGCG), quercetin, and curcumin.
Aspect 35. The method of Aspect 22, wherein the second substance upregulates CAMP gene expression by activating TLR4.
Aspect 36. The method of Aspect 22, wherein the second substance upregulates CAMP gene expression by activating TLR2.
Aspect 37. The method of Aspect 22, wherein the target range of the dysbiosis biomarker is based on a reference range of the dysbiosis biomarker in healthy individuals.
Aspect 38. The method of Aspect 22, wherein the target range of the dysbiosis biomarker is based on a predetermined threshold of the dysbiosis biomarker associated with a reduced risk of disease or improved health outcomes.
Aspect 39. The method of Aspect 22, wherein the composition further comprises a prebiotic or a probiotic.
Aspect 40. The method of Aspect 22, wherein the composition further comprises a non-polyphenol antioxidant.
Aspect 41. The method of Aspect 22, wherein the composition further comprises a non-polyphenol anti-inflammatory agent.
Aspect 42. The method of Aspect 22, wherein the composition further comprises a non-polyphenol immunomodulatory agent.
Aspect 43. The method of Aspect 22, wherein the subject is a mammal.
Aspect 44. The method of Aspect 22, wherein the subject is a human.
Aspect 45. The method of Aspect 22, wherein the dysbiosis is associated with a gastrointestinal disorder.
Aspect 46. The method of Aspect 22, wherein the dysbiosis is associated with an autoimmune disorder.
Aspect 47. The method of Aspect 22, wherein the dysbiosis is associated with a metabolic disorder.
Aspect 48. The method of Aspect 22, wherein the dysbiosis is associated with a neurodegenerative disorder.
Aspect 49. The method of Aspect 22, wherein the dysbiosis is associated with a cardiovascular disorder.
Aspect 50. A method of determining whether an individual has an abnormal level of zonulin in their blood serum, comprising:
   (a) ascertaining blood serum levels (Z_{bs}) of zonulin in the individual;
   (b) comparing the ascertained Z_{bs} to blood serum levels of zonulin in a control group (Z_{c}) comprising healthy, age-matched or sex-matched individuals, thereby determining Δ_{bs}=|Z_{bs}−Z_{c}|, wherein the control group may include relatives of the individual;
   (c) administering a composition comprising a polyphenol and a material which induces CAMP gene expression to the individual; and
   (d) repeating steps a-c until Δ_{bs} is within a predetermined range.
Aspect 51. The method of Aspect 50, wherein the polyphenol is selected from the group consisting of green tea extract, resveratrol, quercetin, and curcumin.
Aspect 52. The method of Aspect 50, wherein the material which induces CAMP gene expression is selected from the group consisting of epinephrine, norepinephrine, and forskolin.
Aspect 53. The method of Aspect 50, wherein the predetermined range of Δ_{bs} is between 5% and 20%.
Aspect 54. The method of Aspect 50, wherein the composition comprising a polyphenol and a material which induces CAMP gene expression is administered orally.
Aspect 55. The method of Aspect 50, wherein the control group is selected from the group consisting of healthy individuals, individuals of the same age as the individual being tested, and individuals of the same sex as the individual being tested.
Aspect 56. The method of Aspect 50, wherein the composition further comprises a probiotic.
Aspect 57. The method of Aspect 56, wherein the probiotic contains at least one bacteria selected from the group consisting of Lactobacillus acidophilus, Bifidobacterium bifidum, and Streptococcus thermophilus.
Aspect 58. The method of Aspect 50, wherein the administering of the composition comprising a polyphenol and a material which induces CAMP gene expression is carried out in conjunction with dietary changes.
Aspect 59. The method of Aspect 58, wherein the dietary changes comprise a reduction in consumption of gluten-containing foods.
Aspect 60. The method of Aspect 50, wherein the abnormal level of zonulin is indicative of a disease or disorder selected from the group consisting of celiac disease, Crohn's disease, and leaky gut syndrome.
Aspect 61. The method of any one of Aspects 50-60, wherein the Δ_{bs} is determined using a quantitative assay.
Aspect 62. The method of any one of Aspects 50-61, wherein the polyphenol is present in the composition at a concentration of 50-1000 mg per dose.
Aspect 63. The method of any one of Aspects 50-62, wherein the material which induces CAMP gene expression is present in the composition at a concentration of 1-10 μM.
Aspect 64. The method of any one of Aspects 50-63, wherein the predetermined range of Δ_{bs} is adjusted based on the individual's medical history.
Aspect 65. The method of any one of Aspects 50-64, wherein the abnormal level of zonulin is indicative of a condition selected from the group consisting of food allergies, autoimmune disorders, and inflammatory bowel disease.
Aspect 66. The method of any one of Aspects 50-65, further comprising correlating the Δ_{bs} with the severity of the condition being treated.
Aspect 67. A method of treating an individual with an abnormal level of zonulin in their blood serum, comprising:
   monitoring the blood serum levels of zonulin in the individual; and
   administering to the individual, during the monitoring, a composition comprising a polyphenol and a material which induces CAMP gene expression to the individual until the difference between blood serum levels of zonulin in the subject and those of a reference control are not statistically significant.
Aspect 68. The method of Aspect 67, wherein the polyphenol is selected from the group consisting of green tea extract, resveratrol, quercetin, and curcumin.
Aspect 69. The method of Aspect 67, wherein the material which induces CAMP gene expression is selected from the group consisting of epinephrine, norepinephrine, and forskolin.
Aspect 70. The method of Aspect 67, wherein the composition comprising a polyphenol and a material which induces CAMP gene expression is administered orally.
Aspect 71. The method of Aspect 67, wherein the reference control is selected from the group consisting of healthy individuals, individuals of the same age as the individual being tested, and individuals of the same sex as the individual being tested.
Aspect 72. The method of Aspect 67, wherein the composition further comprises a probiotic.
Aspect 73. The method of Aspect 72 wherein the probiotic contains at least one bacteria selected from the group consisting of Lactobacillus acidophilus, Bifidobacterium bifidum, and Streptococcus thermophilus.
Aspect 74. The method of Aspect 67, wherein the administering of the composition comprising a polyphenol and a material which induces CAMP gene expression is carried out in conjunction with dietary changes.
Aspect 75. The method of Aspect 74, wherein the dietary changes comprise a reduction in consumption of gluten-containing foods.
Aspect 76. The method of Aspect 67, wherein the abnormal level of zonulin is indicative of a disease or disorder selected from the group consisting of celiac disease, Crohn's disease, and leaky gut syndrome.
Aspect 77. The method of any one of Aspects 67-76, wherein the difference between blood serum levels of zonulin in the subject and those of a reference control (Abs) is determined using a quantitative assay.
Aspect 78. The method of any one of Aspects 67-77, wherein the polyphenol is present in the composition at a concentration of 50-1000 mg per dose.
Aspect 79. The method of any one of Aspects 67-78, wherein the material which induces CAMP gene expression is present in the composition at a concentration of 1-10 M.
Aspect 80. The method of any one of Aspects 67-79, wherein the difference between blood serum levels of zonulin in the subject and those of a reference control (Δ_{bs}) is adjusted based on the individual's medical history.
Aspect 81. The method of any one of Aspects 67-80, wherein the abnormal level of zonulin is indicative of a condition selected from the group consisting of food allergies, autoimmune disorders, and inflammatory bowel disease.
Aspect 82. The method of any one of Aspects 67-81, further comprising correlating the difference between blood serum levels of zonulin in the subject and those of a reference control (Δ_{bs}) with the severity of the condition being treated.
Aspect 83. A method for assessing the integrity of tight junction (TJ) barriers in a subject, comprising:
   measuring the presence, concentration or amount of at least one biomarker in a biological specimen obtained from the subject, wherein the at least one biomarker correlates with TJ barrier integrity; and
   comparing the measured presence, concentration or amount of the at least one biomarker to a reference value to determine the TJ barrier integrity of the subject.
Aspect 84. The method of Aspect 83, wherein the biological specimen is selected from the group consisting of blood, serum, plasma, urine, saliva, and cerebrospinal fluid.
Aspect 85. The method of Aspect 83, wherein the at least one biomarker is selected from the group consisting of claudin-1, occludin, ZO-1, and JAM-A.
Aspect 86. The method of Aspect 83, wherein the at least one biomarker is a protein.
Aspect 87. The method of Aspect 83, wherein the at least one biomarker is an mRNA.
Aspect 88. The method of Aspect 83, wherein the at least one biomarker is a miRNA.
Aspect 89. The method of Aspect 83, wherein the at least one biomarker is an exosome.
Aspect 90. The method of Aspect 83, further comprising:
administering a therapeutic agent to the subject based on the TJ barrier integrity determined by the measured concentration or amount of the at least one biomarker.
Aspect 91. The method of Aspect 90, wherein the therapeutic agent is selected from the group consisting of anti-inflammatory agents, anti-tumor agents, and anti-infective agents.
Aspect 92. The method of Aspect 83, wherein the subject is a human.
Aspect 93. The method of Aspect 83, wherein the subject has a disease or condition associated with TJ barrier dysfunction.
Aspect 94. The method of Aspect 93, wherein the disease or condition is selected from the group consisting of inflammatory bowel disease, irritable bowel syndrome, leaky gut syndrome, celiac disease, and asthma.
Aspect 95. The method of Aspect 83, wherein the measurement of the at least one biomarker is performed using a method selected from the group consisting of ELISA, Western blotting, qPCR, and next-generation sequencing.
Aspect 96. The method of Aspect 83, wherein the measurement of the at least one biomarker is performed using a biosensor.
Aspect 97. The method of Aspect 82, wherein the TJ barrier integrity is determined by measuring the presence, concentration, or amount of a panel of biomarkers.
Aspect 98. The method of any one of Aspects 83-976, wherein the at least one biomarker comprises occludin.
Aspect 99. The method of any one of Aspects 83-97, wherein the biological specimen is selected from the group consisting of blood, urine, saliva, and cerebrospinal fluid.
Aspect 100. The method of any one of Aspects 83-97, wherein the subject is at risk for or has a disease or disorder associated with TJ barrier dysfunction.
Aspect 101. The method of any one of Aspects 83-97, wherein the at least one biomarker is measured using an immunoassay.
Aspect 102. The method of any one of Aspects 83-97, further comprising administering a therapeutic agent to the subject based on the measured TJ barrier integrity.
Aspect 103. The method of any one of Aspects 83-102, further comprising administering a therapeutic agent to the subject comprising a polyphenol in an amount effective to increase TJ barrier integrity.
Aspect 104. The method of Aspect 103, wherein the polyphenol is selected from the group consisting of resveratrol, quercetin, epigallocatechin gallate (EGCG), and curcumin.
Aspect 105. The method of Aspect 103, wherein the polyphenol is administered orally, topically, or intravenously.
Aspect 106. The method of Aspect 103, wherein the polyphenol is administered in a sustained-release formulation.
Aspect 107. The method of Aspect 103, wherein the polyphenol is administered orally.
Aspect 108. The method of Aspect 103, wherein the polyphenol is administered in combination with at least one other compound selected from the group consisting of probiotics, prebiotics, antibiotics, anti-inflammatory agents, and anti-cancer agents.
Aspect 109. The method of Aspect 103, wherein the at least one other compound is administered simultaneously with the polyphenol.
Aspect 110. The method of Aspect 103, wherein the at least one other compound is administered sequentially with the polyphenol.
Aspect 111. The method of Aspect 103, wherein the at least one other compound is administered at a different site than the polyphenol.
Aspect 112. The method of Aspect 103, wherein the polyphenol is administered in a pharmaceutical composition comprising a pharmaceutically acceptable carrier.
Aspect 113. The method of Aspect 103, wherein the polyphenol is administered in a nutraceutical composition comprising a food-grade carrier.
Aspect 114. The method of Aspect 102, wherein the polyphenol is administered in a functional food composition comprising a food-grade carrier.
Aspect 115. The method of Aspect 103, wherein the polyphenol is encapsulated in a liposome, nanoparticle, or microsphere.
Aspect 116. A method of treating a subject, comprising:
   ascertaining a microbiota index measured in a sample of subgingival fluid from the individual;
   comparing the microbiota index to a reference value determined through statistical analysis or machine learning of the preponderance of microbial genera or species in subgingival fluid from healthy and diseased subjects;
   determining the degree of oral dysbiosis in the individual based on the comparison; and
   administering a composition to the subject until the degree of oral dysbiosis is within a predetermined range;
   wherein the composition comprises a polyphenol and a material which induces CAMP gene expression in the individual.
Aspect 117. The method of Aspect 116, wherein the microbiota index is ascertained by performing a DNA sequencing analysis on the sample of subgingival fluid.
Aspect 118. The method of Aspect 116, wherein the reference value is determined through machine learning algorithms trained on a dataset comprising microbiota information from a plurality of healthy and diseased subjects.
Aspect 119. The method of Aspect 116, wherein the degree of oral dysbiosis is determined by calculating a dysbiosis index based on the comparison of the microbiota index and the reference value.
Aspect 120. The method of Aspect 116, wherein the composition further comprises an antimicrobial agent selected from the group consisting of chlorhexidine, cetylpyridinium chloride, and triclosan.
Aspect 121. The method of Aspect 116, wherein the composition further comprises a probiotic microorganism selected from the group consisting of Lactobacillus acidophilus, Bifidobacterium bifidum, and Streptococcus thermophilus.
Aspect 122. The method of Aspect 116, wherein the composition is administered to the subject via oral application.
Aspect 123. The method of Aspect 116, wherein the predetermined range of oral dysbiosis is determined based on the subject's medical history, age, gender, and lifestyle factors.
Aspect 124. The method of Aspect 116, wherein the composition is administered to the subject in conjunction with a dental procedure selected from the group consisting of scaling, root planing, and periodontal surgery.
Aspect 125. The method of any one of Aspects 116-124, wherein the polyphenol is selected from the group consisting of epigallocatechin-3-gallate (EGCG), resveratrol, quercetin, and curcumin.
Aspect 126. The method of any one of Aspects 116-125, wherein the material which induces CAMP gene expression is selected from the group consisting of vitamin D, butyrate, and β-glucan.
Aspect 127. The method of any one of Aspects 116-126, wherein the composition is administered to the subject in a sustained-release form.
Aspect 128. The method of any one of Aspects 116-127, wherein the composition is administered to the subject in a combination therapy with an antibiotic.
Aspect 129. The method of any one of Aspects 116-128, wherein the subject has a history of periodontal disease.
Aspect 130. The method of any one of Aspects 116-129, wherein the degree of oral dysbiosis is monitored over time to adjust the dosage and duration of the composition administration.
Aspect 131. The method of any one of Aspects 116-130, wherein the composition is administered to the subject in combination with an oral hygiene regimen consisting of tooth brushing and flossing.
Aspect 132. A method of treating a subject, comprising:
   ascertaining a microbiota index measured in a sample taken from a source microbiome selected from the group consisting of the gut, skin, urinary, aural, ocular, genital, pulmonary, nasopharyngeal, tonsillar, and umbilical microbiomes;
   comparing the microbiota index to a reference value determined through statistical analysis or machine learning of the preponderance of microbial genera or species in a sample from healthy and diseased subjects in the source microbiome;
   determining the degree of dysbiosis in the source microbiome based on the comparison; and
   administering a composition to the subject until the degree of dysbiosis in the source microbiome is within a predetermined range;
   wherein the composition comprises a polyphenol and a material which induces CAMP gene expression in the individual.
Aspect 133. The method of Aspect 132, wherein the ocular microbiome includes microorganisms inhabiting the surface of the eye and surrounding structures, including the conjunctiva, eyelids, and tear ducts.
Aspect 134. The method of Aspect 132, wherein the microbiota index is ascertained by performing a DNA sequencing analysis on the sample.
Aspect 135. The method of Aspect 132, wherein the reference value is determined through machine learning algorithms trained on a dataset comprising microbiota information from a plurality of healthy and diseased subjects.
Aspect 136. The method of Aspect 132, wherein the degree of dysbiosis is determined by calculating a dysbiosis index based on the comparison of the microbiota index and the reference value.
Aspect 137. The method of Aspect 132, wherein the composition further comprises an antimicrobial agent selected from the group consisting of chlorhexidine, cetylpyridinium chloride, and triclosan.
Aspect 138. The method of Aspect 132, wherein the composition further comprises a probiotic microorganism selected from the group consisting of Lactobacillus acidophilus, Bifidobacterium bifidum, and Streptococcus thermophilus.
Aspect 139. The method of Aspect 132, wherein the composition is administered to the subject via oral application.
Aspect 140. The method of Aspect 132, wherein the composition is administered to the subject via topical application.
Aspect 141. The method of Aspect 132, wherein the composition is administered to the subject via transdermal application.
Aspect 142. The method of Aspect 132, wherein the predetermined range of dysbiosis is determined based on the subject's medical history, age, gender, and lifestyle factors.
Aspect 143. The method of any one of Aspects 132-142, wherein the polyphenol is selected from the group consisting of epigallocatechin-3-gallate (EGCG), resveratrol, quercetin, and curcumnin.
Aspect 144. The method of any one of Aspects 132-143, wherein the material which induces CAMP gene expression is selected from the group consisting of vitamin D, butyrate, and (3-glucan.
Aspect 145. The method of any one of Aspects 132-144, wherein the composition is administered to the subject in a sustained-release form.
Aspect 146. The method of any one of Aspects 132-145, wherein the composition is administered to the subject in a combination therapy with an antibiotic.
Aspect 147. The method of any one of Aspects 132-146, wherein the degree of oral dysbiosis is monitored over time to adjust the dosage and duration of the composition administration.
Aspect 148. The method of any one of Aspects 132-147, wherein ascertaining a microbiota index measured in a sample taken from a source microbiome includes ascertaining a first microbiota index measured in a sample taken from a first source microbiome and ascertaining a second microbiota index measured in a sample taken from a second source microbiome, wherein said first and second source microbiomes are distinct.
Aspect 149. The method of Aspect 148, wherein comparing the microbiota index to a reference value includes comparing the first microbiota index to a first reference value and comparing the second microbiota index to a second reference value.
Aspect 150. The method of Aspect 149, wherein determining the degree of dysbiosis includes performing multivariate analysis based on the first and second microbiota indices.
Aspect 151. The method of Aspect 150, wherein determining the degree of dysbiosis includes performing multivariate analysis based on the first and second reference values.

### EXPERIMENTAL

The following examples are offered by way of illustration and not by way of limitation.

### Example 1: Polyphenolic Compositions

Some embodiments of the systems and methodologies described herein may utilize polyphenol phytosomes, that is, complexes where the polyphenol is bound to phospholipids to enhance its absorption and bioavailability. The use of such complexes may increase the effectiveness of the polyphenol in some applications by improving its ability to pass through cell membranes and enter the bloodstream. This may result in potentially better therapeutic effects for some conditions. This is especially true of polyphenols having low solubility under physiological conditions, which may adversely impact their absorption and bioavailability. The use of quercetin phytosome, curcumin phytosome, and green tea phytosome for these purposes is especially preferred. These compositions are commercially available from Thorne Research Inc. (New York, NY)).

This example illustrates the preparation of a composition in accordance with the teachings herein.

A composition was prepared having the components depicted in TABLE 3. The composition was formulated by mixing appropriate amounts of Advanced DHA, PolyResveratrol-SR and 1,25-dihydroxyvitamin D3, all obtained commercially from Thorne Research Inc., (New York, NY), in 2-Hydroxypropyl-β-cyclodextrin.

TABLE 3
Compound Formulations

| Compound | Dose |
|---|---|
| (Vehicle = TBD % 2-Hydroxypropyl-β-cyclodextrin) | (mg/kg) |
| Docosahexaenoic Acid (DHA) ^{A} | 133.25 |
| Eicosapentaenoic Acid (EPA) ^{A} | 41 |
| Omega-3 Fatty Acids (EPA/DHA) ^{A} | 196.8 |
| Curcumin Phytosome (Meriva) ^{B} | 20.5 |
| Green Tea Phytosome (decaffeinated) ^{B} | 20.5 |
| Quercetin Phytosome ^{B} | 20.5 |
| Trans-Pterostilbene ^{B} | 20.5 |
| Trans-Resveratrol ^{B} | 20.5 |
| 1,25-dihydroxyvitamin D3 | 0.002 |

^{A} from Thorne Research Advanced DHA
^{B} from Thorne Research PolyResveratrol-SR

### Example 2: Testing Intestinal Permeability Using a Lactulose to Mannitol Ratio (LMR)

This example illustrates how intestinal permeability would be assessed in a patient in a clinical setting using the Lactulose to Mannitol Ratio (LMR).

The patient is instructed to fast for at least 2 hours before the test to ensure an empty stomach. The patient is also instructed to empty their bladder just before consuming the test solution to start the test with an empty urinary tract.

A treatment solution is prepared by mixing lactulose and mannitol in specific concentrations, typically 250 mg/ml for lactulose and 50 mg/ml for mannitol. The treatment solution is administered to the patient orally at a dose of 2 ml per kg of body weight, up to a maximum of 20 ml. This ensures an adequate volume for both children and adults.

A urine collection bag is attached to the patient to collect all urine excreted. Typically, urine is collected for 2 hours post-ingestion. A 5-hour collection period can also be used based on the clinical judgement of the patient's condition or comparative studies.

A preservative such as thimerosal is added to the collected urine immediately to prevent degradation of the sugars. The urine samples are then stored at −80° C. if not analyzed immediately to preserve the integrity of the samples.

At the time of analysis, the samples are thawed and prepared by vortexing and centrifuging to clarify. This preparation may be crucial for accurate analysis. HPAE-PAD or LC-MS/MS techniques are then employed to quantitatively measure the concentrations of lactulose and mannitol in the urine. Suitable calibration standards are utilized to ensure the accuracy and precision of the measurements.

The Lactulose to Mannitol Ratio (LMR) is then calculated. The LMR ratio in the urine reflects intestinal permeability. A higher LMR indicates greater permeability, suggesting a compromised barrier function. By way of reference, a normal LMR, indicative of healthy intestinal permeability, typically ranges from approximately 0.01 to 0.03. This ratio reflects a balance where mannitol, a small molecule that is easily absorbed, is found in higher concentrations compared to lactulose, a larger molecule that is poorly absorbed and primarily passes through the gut. An elevated LMR (above 0.03) suggests increased intestinal permeability, often referred to as “leaky gut.” This can occur in various conditions such as inflammatory bowel disease, celiac disease, and intestinal infections. Conversely, a very low LMR might indicate issues with nutrient absorption. However, it is important to note that the “normal” range can vary based on the methodology used and the specific population being tested, so clinicians will often refer to the reference ranges provided by the specific laboratory performing the test.

### Example 3: Testing Intestinal Permeability Using Zonulin

This example illustrates how intestinal permeability would be assessed in a patient in a clinical setting using zonulin as a biomarker of tight junction permeability.

Zonulin levels can be measured in both blood and feces, with fecal testing being more commonly used due to its non-invasive nature and direct association with gut permeability. A blood draw would be performed if serum zonulin levels are to be measured. More commonly, the patient would be given instructions on how to collect a stool sample properly to avoid contamination and ensure accurate results.

The collected samples would be sent to a laboratory experienced in conducting zonulin tests. These tests typically involve enzyme-linked immunosorbent assays (ELISA) designed to quantitatively measure zonulin levels. Examples include the IDK® Zonulin (serum) ELISA kit or the IDK® Zonulin (stool) ELISA kit available from Immundiagnostik AG, Bensheim, Germany.

Once the test results are available, the clinician would interpret them based on current research and reference values. Elevated zonulin levels can suggest increased intestinal permeability, often referred to as “leaky gut.” This condition allows substances that should be contained within the gastrointestinal tract to pass into the bloodstream, potentially leading to various health issues. For reference, normal levels of zonulin in the blood are typically considered to be below 30 ng/mL, with values higher than this being indicative of increased intestinal permeability. For zonulin measured in stool, the normal range is less well defined and can vary more widely, but a normal range is often considered to be less than 78 ng/mL.

### Example 4: Treating Leaky Gut Syndrome by Inducing CAMP Gene Expression

This example illustrates how compositions inducing CAMP gene expression could be used in a clinical setting to treat patients suffering from leaky gut syndrome or other issues related to intestinal permeability. This example assumes that the protocol described in EXAMPLE 1 was followed and that LMR levels indicate abnormal intestinal permeability in the patient.

A treatment involving a CAMP gene expression inducing composition is chosen. In this example, the composition is a combination of orally administered capsules and droplets. The capsules contain 250 mg quercetin phytosome each disposed in a hypromellose capsule, and also contain leucine, microcrystalline cellulose and silicon dioxide. The droplets contain 25 mcg vitamin D3 per drop and also include medium chain triglycerides and mixed tocopherols. The composition is typically administered at a daily dosage of one capsule and 2 droplets, although the clinician may vary the initial dosage taking into account factors such as the patient's condition, age, weight, and overall health.

Regular follow-up visits are scheduled with the patient to monitor the patient's response to the treatment through clinical assessment and repeated lactulose-mannitol tests. These follow-ups may be monthly or bimonthly, depending on the initial severity of the condition and the patient's response to the treatment. Lactulose-mannitol tests are repeated at designated intervals (e.g., every 3-6 months) to quantitatively assess changes in intestinal permeability. Based on the results of the lactulose-mannitol tests, the treatment dosage is adjusted as appropriate to optimize the therapeutic outcome. For example, if LMR levels are found to be at physiologically healthy levels, administration of the treatment composition may be suspended.

### Example 5: Assessing Gut Dysbiosis Using the Firmicutes/Bacteroidetes Ratio

This example illustrates a clinical procedure for assessing gut dysbiosis in a clinical setting using the Firmicutes/Bacteroidetes Ratio.

The patient is instructed to avoid any dietary changes or antibiotics for a period (typically a few weeks) prior to the sample collection, as these can affect the microbiome composition. A fecal sample is then collected from the patient using a sterile collection kit. The patient is provided with all necessary materials and instructions for proper collection to avoid contamination.

Microbial DNA is then extracted from the fecal sample using a method that involves the steps of sample homogenization, cell lysis, and DNA purification. These steps are described in further detail below.

Sample homogenization aims to uniformly disrupt microbial cells and ensure consistent DNA recovery. This step involves mechanical homogenization, typically using bead-beating techniques, where the sample is vigorously shaken with small beads in a homogenizer to break down the cells effectively. The process uses bead mill equipment and sterile beads made of materials like glass or ceramic, chosen for their ability to lyse cells without damaging the DNA. Critical parameters such as the speed and duration of shaking, as well as the sample-to-bead ratio, are carefully optimized to maximize cell disruption while minimizing DNA shearing. Proper homogenization creates a uniform cell lysate, setting the stage for efficient subsequent lysis and DNA extraction steps, thereby enhancing the overall quality and yield of the extracted DNA.

Cell lysis is performed by adding a lysis buffer containing a detergent (here, SDS) to the homogenized sample to further disrupt the cell membranes. Enzymes such as lysozyme, Proteinase K, and RNase are added to the lysis buffer to aid in breaking down cell walls (especially for Gram-positive bacteria) and degrading proteins and RNA, respectively. The mixture is then incubated at an appropriate temperature (usually around 56° C.) for a period (typically 1-2 hours) to ensure complete lysis of cells.

The DNA purification step in microbial DNA extraction from fecal samples involves isolating DNA from the cell lysate while removing impurities such as proteins, lipids, and polysaccharides. This is typically achieved using DNA binding columns or magnetic beads that selectively bind DNA under certain salt and pH conditions. After transferring the lysate to these binding systems, a series of washes with a wash buffer are performed to remove any unbound materials. Finally, the purified DNA is eluted from the binding medium using an elution buffer or water, which detaches the DNA from the column or beads, allowing it to be collected in a new tube. The effectiveness of this step is crucial as it determines the purity and concentration of DNA, which are vital for accurate and reliable downstream genetic analysis. This step also ensures that the DNA is free from contaminants that could interfere with PCR amplification and sequencing processes.

DNA quantification and quality assessment step is typically crucial for evaluating the yield and purity of extracted DNA, ensuring it is suitable for downstream applications such as sequencing. This step typically involves using spectrophotometric methods to measure DNA concentration, where the absorbance at 260 nm gives an indication of DNA quantity, while the A260/A280 ratio helps assess purity, with a ratio around 1.8 indicating relatively pure DNA free of protein contamination. Fluorometric methods can also be employed, using fluorescent dyes that bind specifically to DNA, providing a more sensitive and accurate quantification. Additionally, the integrity of the DNA is checked using agarose gel electrophoresis, which allows visualization of the DNA size distribution; intact DNA will appear as a clear, distinct band, whereas degraded DNA may show smearing. This comprehensive assessment ensures that the DNA extracted is of high quality and quantity, critical for reliable analytical results in molecular biology studies.

It is to be noted that several commercial kits are available that streamline the DNA extraction process from fecal samples, such as the QIAamp DNA Stool Mini Kit (Qiagen) and the PowerSoil DNA Isolation Kit (Mo Bio). These kits are designed to handle the complexities of fecal samples and often provide more consistent results than homebrew methods.

Microbial DNA sequencing is then performed on the sample, which involves the amplification and sequencing of 16S rRNA genes. This step plays a pivotal role in analyzing the microbial communities in fecal samples. The process begins with the selection of specific primers that target conserved regions of the 16S rRNA gene shared by key bacterial groups like Firmicutes and Bacteroidetes. These primers are used in a polymerase chain reaction (PCR) to selectively amplify these regions, which are then prepared for sequencing by attaching platform-specific adapters to the amplified products.

For sequencing, advanced next-generation sequencing (NGS) platforms such as Illumina or PacBio are employed. Illumina is favored for its high-throughput capability and short-read accuracy, ideal for distinguishing bacterial taxa at the species level by targeting specific hypervariable regions of the 16S rRNA gene. In contrast, PacBio provides longer reads that can cover the entire 16S rRNA gene, offering deeper phylogenetic insights and the ability to differentiate closely related microbial species.

Following sequencing, the data undergoes rigorous processing. Initial steps involve filtering out low-quality reads to ensure the integrity of the analysis. High-quality reads are then aligned against known 16S rRNA gene sequences in reference databases to identify and classify microbial taxa. This alignment helps in identifying operational taxonomic units (OTUs) or amplicon sequence variants (ASVs). OTUs group sequences based on a similarity threshold, usually set at 97%, to cluster sequences that likely represent the same species. ASVs provide a finer resolution by distinguishing sequences that differ by even a single nucleotide, allowing for a more precise identification of microbial taxa.

The sequences are then taxonomically classified based on their best matches in the reference database, determining which sequences belong to Firmicutes, Bacteroidetes, or other groups. The final step in the data processing is the calculation of the relative abundance and diversity of these groups, which sheds light on the structure and health of the microbial community. This comprehensive approach to sequencing and data analysis is essential for accurately assessing microbial diversity and understanding the implications of microbial imbalances in clinical settings.

Quantifying the relative abundance of the bacterial phyla Firmicutes and Bacteroidetes from microbial DNA sequencing data involves several crucial steps. After the DNA extraction and sequencing processes, the data obtained is comprised of sequences that can be specifically attributed to different bacterial taxa based on their unique genetic markers, in this case, regions of the 16S rRNA gene. The sequences are first aligned and compared against a reference database to identify and classify each sequence to its corresponding bacterial group. Advanced bioinformatics tools and software may be used to analyze the read counts associated with each identified bacterial phylum.

The relative abundance of each phylum is then calculated by determining the proportion of sequences (or read counts) that align with Firmicutes and those that align with Bacteroidetes out of the total bacterial sequences obtained from the sample. This provides a percentage representation of each group within the total microbial community, offering insights into the dominance or lack thereof of these groups within the gut flora.

Following the determination of relative abundances, the ratio of Firmicutes to Bacteroidetes is computed, which is a critical indicator often used in microbial community studies related to human health. This ratio is calculated by dividing the total abundance (or percentage) of Firmicutes by the total abundance of Bacteroidetes. For instance, if the sequencing results show that 60% of the identified bacteria belong to Firmicutes and 30% to Bacteroidetes, the F/B ratio would be 2:1. This ratio provides a quantitative measure that may be utilized to evaluate microbial balance or dysbiosis in the gut. A higher or lower F/B ratio can be indicative of various health states or conditions, such as obesity, diabetes, or inflammatory bowel disease, and is therefore crucial for diagnostic and therapeutic considerations. Additional factors,

The F/B ratio obtained from the patient's fecal sample is the compared to a suitable sample population. The suitable sample population for a particular patient may be based on considerations such as age, geographic or ethnic background, diet, health status (for example, whether the patient is diabetic or overweight), lifestyle factors, and antibiotic usage. For example, in one sample population of obese subjects, an average F/B ratio was found to be 0.9, meaning the amount of Firmicutes was nearly equal to but slightly lower than Bacteroidetes. For comparison, in one sample population of lean subjects, an average F/B ratio was found to be 0.4, indicating a greater abundance of Bacteroidetes relative to Firmicutes. See Ley, R. E., Turnbaugh, P. J., Klein, S., & Gordon, J. I. (2006). Microbial ecology: Human gut microbes associated with obesity. Nature, 444(7122), 1022-1023. doi:10.1038/4441022a.

While the foregoing example focuses specifically on use of the F/B ratio, it will be appreciated that clinical application of the foregoing method may consider other characteristics of the gut microbiome to provide a more nuanced view of the patient's gut microbiota.

For example, various diversity indices may be considered such as alpha diversity and beta diversity. Reduced alpha diversity, which reflects fewer species and less evenness in species distribution, has been associated with various health issues, such as inflammatory bowel disease (IBD) and obesity. Specific numerical thresholds for indices like Shannon or Simpson diversity scores that define dysbiosis are not universally established but are often significantly lower in diseased states compared to healthy controls. Significant shifts in community composition (beta diversity) compared to healthy controls can indicate dysbiosis. Methods such as UniFrac distances or Bray-Curtis dissimilarity may be used to quantify these shifts, but specific cutoff points are typically study-specific.

Specific microbial ratios may also be considered. In addition to the F/B ratio described above, these may include, for example, ratios of protective to pathogenic bacteria, such as Faecalibacterium prausnitzii (a protective species) relative to Escherichia coli (potentially pathogenic), can serve as potential indicators.

Finally, metabolite levels may also be informative. Reduced concentrations of SCFAs such as butyrate, propionate, and acetate in the stool are often indicative of dysbiosis because these are major products of bacterial fermentation beneficial for colon health. Specific concentration thresholds may vary but are typically lower in patients with conditions like IBD. Elevated levels of p-cresol and ammonia in the urine or feces have been associated with an overgrowth of certain pathogenic bacteria and can indicate protein fermentation rather than carbohydrate fermentation (which is healthier).

Accordingly, the preceding merely illustrates the principles of the present disclosure. It will be appreciated that those skilled in the art will be able to devise various arrangements which, although not explicitly described or shown herein, embody the principles of the invention and are included within its spirit and scope, including additions, substitutions, and modifications to the foregoing embodiments. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Accordingly, the scope of the present invention should be construed in reference to the appended claims. It will also be appreciated that the various features set forth in the claims may be presented in various combinations and sub-combinations in future claims without departing from the scope of the invention. In particular, the present disclosure expressly contemplates any such combination or sub-combination that is not known to the prior art, as if such combinations or sub-combinations were expressly written out.

What is claimed is:

1. A method for modulating tight junction (TJ) integrity in a subject, comprising:
(a) assessing TJ integrity in the subject by quantifying at least one biomarker of TJ integrity in the subject;
(b) administering to the subject a first composition containing
(i) at least one polyphenol, and
(ii) a second substance which is not a polyphenol and which upregulates CAMP gene expression in the subject;
(c) reassessing TJ integrity in the subject by quantifying at least one biomarker of TJ integrity in the subject; and
(d) while the value of said at least one biomarker is outside of a target range, repeating steps (b) and (c).

2. The method of claim 1, wherein assessing TJ integrity in the subject includes quantifying at least one biomarker of TJ integrity in a biological sample obtained from the subject.

3. The method of claim 2, wherein said biological sample is selected from the group consisting of whole blood, blood plasma, and blood serum.

4. The method of claim 1, further comprising:
after the value of said at least one biomarker is within a target range, administering the first composition to the subject on a periodic basis.

5. The method of claim 1, wherein the assessed TJ integrity is of a barrier layer in epithelial cells.

6. The method of claim 1, wherein the assessed TJ integrity is of a barrier layer in endothelial cells.

7. The method of claim 1, wherein the assessed TJ integrity is of the boundary between apical and basolateral membrane domains in epithelial and endothelial cells.

8. The method of claim 1, wherein the assessed TJ integrity is of a barrier layer selected from the group consisting of the blood-brain barrier (BBB) and the intestinal epithelial barrier layer.

9. The method of claim 1, wherein said at least one polyphenol comprises quercetin.

10. The method of claim 9, wherein said quercetin is present as a blend with at least one phospholipid.

11. The method of claim 9, wherein said at least one phospholipid includes lecithin.

12. The method of claim 1, wherein said at least one polyphenol is a flavonoid.

13. The method of claim 12, wherein said flavonoid is hydroxyl substituted 3-hydroxy-2-phenylchromen-4-one.

14. The method of claim 12, wherein said flavonoid is selected from the group consisting of flavonoids and bioflavonoids.

15. The method of claim 12, wherein said flavonoid is selected from the group consisting of isoflavonoids derived from 3-phenylchromen-4-one (3-phenyl-1,4-benzopyrone) structure.

16. The method of claim 12, wherein said flavonoid is selected from the group consisting of neoflavonoids, derived from 4-phenylcoumarine (4-phenyl-1,2-benzopyrone) structure.

17. The method of claim 1, wherein said at least one polyphenol is selected from the group consisting of quercetin, epigallocatechin gallate (EGCG), curcumin, resveratrol, and catechins.

18. The method of claim 1, wherein the second substance is selected from the group consisting of forskolin, histamine, and butyrate.

19. The method of claim 1, wherein the TJ integrity biomarker is selected from the group consisting of occludin, claudin-1, zonula occludens-1 (ZO-1), and junctional adhesion molecule A (JAMA).

20. The method of claim 1, wherein the target range for the TJ integrity biomarker is determined by comparing the biomarker value in the subject to a reference value obtained from a control subject with known TJ integrity.

21. The method of claim 1, wherein the subject has a condition selected from the group consisting of inflammatory bowel disease, irritable bowel syndrome, leaky gut syndrome, and celiac disease.

22. A method for attenuating dysbiosis in a subject, comprising:
(a) assessing a state of dysbiosis in a subject by quantifying at least one dysbiosis biomarker in the subject;
(b) administering to the subject a composition containing
(i) at least one polyphenol, and
(ii) a second substance which is not a polyphenol and which upregulates CAMP gene expression in the subject; and
(c) repeating steps (a) and (b) until the value of said at least one dysbiosis biomarker is within a target range.

23. The method of claim 22, wherein the at least one dysbiosis biomarker is an oral biomarker of dysbiosis.

24. The method of claim 22, wherein the at least one dysbiosis biomarker is a biomarker of dysbiosis in the oral cavity.

25. The method of claim 22, wherein the at least one dysbiosis biomarker is a biomarker of dysbiosis in the colon.

26. The method of claim 22, wherein the at least one dysbiosis biomarker is a biomarker of dysbiosis in the gut.

27. The method of claim 22, wherein the at least one dysbiosis biomarker includes first and second dysbiosis biomarkers, wherein the first and second dysbiosis biomarkers are biomarkers of dysbiosis in distinct regions of the body, and wherein the regions of the body are selected from the group consisting of the mouth, the gut, and the colon.

28. The method of claim 22, wherein the at least one dysbiosis biomarker is an index of subgingival microbial dysbiosis.

29. The method of claim 22, wherein the at least one biomarker is based on the preponderance in a biological sample of at least one periodontitis-associated genera.

30. The method of claim 29, wherein the periodontitis-associated genera is selected from the group consisting of Fretibacterium, Treponema, Mogibacterium, Peptostreptococcaceae genus 6, and Desulfobulbus.

31. The method of claim 22, wherein the at least one biomarker is based on the preponderance in a biological sample of at least one health-associated genera.

32. The method of claim 31, wherein the health-associated genera is selected from the group consisting of Actinomyces and Streptococcus.

33. The method of claim 22, wherein the dysbiosis biomarker is selected from the group consisting of fecal calprotectin, alpha-1-antitrypsin, zonulin, LPS-binding protein, and beta-defensin 2.

34. The method of claim 22, wherein the at least one polyphenol is selected from the group consisting of resveratrol, epigallocatechin gallate (EGCG), quercetin, and curcumin.

35. The method of claim 22, wherein the second substance upregulates CAMP gene expression by activating TLR4.

36. The method of claim 22, wherein the second substance upregulates CAMP gene expression by activating TLR2.

37. The method of claim 22, wherein the target range of the dysbiosis biomarker is based on a reference range of the dysbiosis biomarker in healthy individuals.

38. The method of claim 22, wherein the target range of the dysbiosis biomarker is based on a predetermined threshold of the dysbiosis biomarker associated with a reduced risk of disease or improved health outcomes.

39. The method of claim 22, wherein the composition further comprises a prebiotic or a probiotic.

40. The method of claim 22, wherein the composition further comprises a non-polyphenol antioxidant.

41. The method of claim 22, wherein the composition further comprises a non-polyphenol anti-inflammatory agent.

42. The method of claim 22, wherein the composition further comprises a non-polyphenol immunomodulatory agent.

43. The method of claim 22, wherein the subject is a mammal.

44. The method of claim 22, wherein the subject is a human.

45. The method of claim 22, wherein the dysbiosis is associated with a gastrointestinal disorder.

46. The method of claim 22, wherein the dysbiosis is associated with an autoimmune disorder.

47. The method of claim 22, wherein the dysbiosis is associated with a metabolic disorder.

48. The method of claim 22, wherein the dysbiosis is associated with a neurodegenerative disorder.

49. The method of claim 22, wherein the dysbiosis is associated with a cardiovascular disorder.

50. A method of determining whether an individual has an abnormal level of zonulin in their blood serum, comprising:
(a) ascertaining blood serum levels (Z_{bs}) of zonulin in the individual;
(b) comparing the ascertained Z_{bs} to blood serum levels of zonulin in a control group (Z_{c}) comprising healthy, age-matched or sex-matched individuals, thereby determining Δ_{bs}=|Z_{bs}−Z_{c}|, wherein the control group may include relatives of the individual;
(c) administering a composition comprising a polyphenol and a material which induces CAMP gene expression to the individual; and
(d) repeating steps a-c until Δ_{bs} is within a predetermined range.

51. The method of claim 50, wherein the polyphenol is selected from the group consisting of green tea extract, resveratrol, quercetin, and curcumin.

52. The method of claim 50, wherein the material which induces CAMP gene expression is selected from the group consisting of epinephrine, norepinephrine, and forskolin.

53. The method of claim 50, wherein the predetermined range of Abs is between 5% and 20%.

54. The method of claim 50, wherein the composition comprising a polyphenol and a material which induces CAMP gene expression is administered orally.

55. The method of claim 50, wherein the control group is selected from the group consisting of healthy individuals, individuals of the same age as the individual being tested, and individuals of the same sex as the individual being tested.

56. The method of claim 50, wherein the composition further comprises a probiotic.

57. The method of claim 56, wherein the probiotic contains at least one bacteria selected from the group consisting of Lactobacillus acidophilus, Bifidobacterium bifidum, and Streptococcus thermophilus.

58. The method of claim 50, wherein the administering of the composition comprising a polyphenol and a material which induces CAMP gene expression is carried out in conjunction with dietary changes.

59. The method of claim 58, wherein the dietary changes comprise a reduction in consumption of gluten-containing foods.

60. The method of claim 50, wherein the abnormal level of zonulin is indicative of a disease or disorder selected from the group consisting of celiac disease, Crohn's disease, and leaky gut syndrome.

61. The method of any one of claims 50-60, wherein the Abs is determined using a quantitative assay.

62. The method of any one of claims 50-61, wherein the polyphenol is present in the composition at a concentration of 50-1000 mg per dose.

63. The method of any one of claims 50-62, wherein the material which induces CAMP gene expression is present in the composition at a concentration of 1-10 μM.

64. The method of any one of claims 50-63, wherein the predetermined range of Δ_{bs} is adjusted based on the individual's medical history.

65. The method of any one of claims 50-64, wherein the abnormal level of zonulin is indicative of a condition selected from the group consisting of food allergies, autoimmune disorders, and inflammatory bowel disease.

66. The method of any one of claims 50-65, further comprising correlating the Abs with the severity of the condition being treated.

67. A method of treating an individual with an abnormal level of zonulin in their blood serum, comprising:
monitoring the blood serum levels of zonulin in the individual; and
administering to the individual, during the monitoring, a composition comprising a polyphenol and a material which induces CAMP gene expression to the individual until the difference between blood serum levels of zonulin in the subject and those of a reference control are not statistically significant.

68. The method of claim 67, wherein the polyphenol is selected from the group consisting of green tea extract, resveratrol, quercetin, and curcumin.

69. The method of claim 67, wherein the material which induces CAMP gene expression is selected from the group consisting of epinephrine, norepinephrine, and forskolin.

70. The method of claim 67, wherein the composition comprising a polyphenol and a material which induces CAMP gene expression is administered orally.

71. The method of claim 67, wherein the reference control is selected from the group consisting of healthy individuals, individuals of the same age as the individual being tested, and individuals of the same sex as the individual being tested.

72. The method of claim 67, wherein the composition further comprises a probiotic.

73. The method of claim 72 wherein the probiotic contains at least one bacteria selected from the group consisting of Lactobacillus acidophilus, Bifidobacterium bifidum, and Streptococcus thermophilus.

74. The method of claim 67, wherein the administering of the composition comprising a polyphenol and a material which induces CAMP gene expression is carried out in conjunction with dietary changes.

75. The method of claim 74, wherein the dietary changes comprise a reduction in consumption of gluten-containing foods.

76. The method of claim 67, wherein the abnormal level of zonulin is indicative of a disease or disorder selected from the group consisting of celiac disease, Crohn's disease, and leaky gut syndrome.

77. The method of any one of claims 67-76, wherein the difference between blood serum levels of zonulin in the subject and those of a reference control (Δ_{bs}) is determined using a quantitative assay.

78. The method of any one of claims 67-77, wherein the polyphenol is present in the composition at a concentration of 50-1000 mg per dose.

79. The method of any one of claims 67-78, wherein the material which induces CAMP gene expression is present in the composition at a concentration of 1-10 M.

80. The method of any one of claims 67-79, wherein the difference between blood serum levels of zonulin in the subject and those of a reference control (Abs) is adjusted based on the individual's medical history.

81. The method of any one of claims 67-80, wherein the abnormal level of zonulin is indicative of a condition selected from the group consisting of food allergies, autoimmune disorders, and inflammatory bowel disease.

82. The method of any one of claims 67-81, further comprising correlating the difference between blood serum levels of zonulin in the subject and those of a reference control (Abs) with the severity of the condition being treated.

83. A method for assessing the integrity of tight junction (TJ) barriers in a subject, comprising:
measuring the presence, concentration or amount of at least one biomarker in a biological specimen obtained from the subject, wherein the at least one biomarker correlates with TJ barrier integrity; and
comparing the measured presence, concentration or amount of the at least one biomarker to a reference value to determine the TJ barrier integrity of the subject.

84. The method of claim 83, wherein the biological specimen is selected from the group consisting of blood, serum, plasma, urine, saliva, and cerebrospinal fluid.

85. The method of claim 83, wherein the at least one biomarker is selected from the group consisting of claudin-1, occludin, ZO-1, and JAM-A.

86. The method of claim 83, wherein the at least one biomarker is a protein.

87. The method of claim 83, wherein the at least one biomarker is an mRNA.

88. The method of claim 83, wherein the at least one biomarker is a miRNA.

89. The method of claim 83, wherein the at least one biomarker is an exosome.

90. The method of claim 83, further comprising:
administering a therapeutic agent to the subject based on the TJ barrier integrity determined by the measured concentration or amount of the at least one biomarker.

91. The method of claim 90, wherein the therapeutic agent is selected from the group consisting of anti-inflammatory agents, anti-tumor agents, and anti-infective agents.

92. The method of claim 83, wherein the subject is a human.

93. The method of claim 83, wherein the subject has a disease or condition associated with TJ barrier dysfunction.

94. The method of claim 93, wherein the disease or condition is selected from the group consisting of inflammatory bowel disease, irritable bowel syndrome, leaky gut syndrome, celiac disease, and asthma.

95. The method of claim 83, wherein the measurement of the at least one biomarker is performed using a method selected from the group consisting of ELISA, Western blotting, qPCR, and next-generation sequencing.

96. The method of claim 83, wherein the measurement of the at least one biomarker is performed using a biosensor.

97. The method of claim 82, wherein the TJ barrier integrity is determined by measuring the presence, concentration, or amount of a panel of biomarkers.

98. The method of any one of claims 83-976, wherein the at least one biomarker comprises occludin.

99. The method of any one of claims 83-97, wherein the biological specimen is selected from the group consisting of blood, urine, saliva, and cerebrospinal fluid.

100. The method of any one of claims 83-97, wherein the subject is at risk for or has a disease or disorder associated with TJ barrier dysfunction.

101. The method of any one of claims 83-97, wherein the at least one biomarker is measured using an immunoassay.

102. The method of any one of claims 83-97, further comprising administering a therapeutic agent to the subject based on the measured TJ barrier integrity.

103. The method of any one of claims 83-102, further comprising administering a therapeutic agent to the subject comprising a polyphenol in an amount effective to increase TJ barrier integrity.

104. The method of claim 103, wherein the polyphenol is selected from the group consisting of resveratrol, quercetin, epigallocatechin gallate (EGCG), and curcumin.

105. The method of claim 103, wherein the polyphenol is administered orally, topically, or intravenously.

106. The method of claim 103, wherein the polyphenol is administered in a sustained-release formulation.

107. The method of claim 103, wherein the polyphenol is administered orally.

108. The method of claim 103, wherein the polyphenol is administered in combination with at least one other compound selected from the group consisting of probiotics, prebiotics, antibiotics, anti-inflammatory agents, and anti-cancer agents.

109. The method of claim 103, wherein the at least one other compound is administered simultaneously with the polyphenol.

110. The method of claim 103, wherein the at least one other compound is administered sequentially with the polyphenol.

111. The method of claim 103, wherein the at least one other compound is administered at a different site than the polyphenol.

112. The method of claim 103, wherein the polyphenol is administered in a pharmaceutical composition comprising a pharmaceutically acceptable carrier.

113. The method of claim 103, wherein the polyphenol is administered in a nutraceutical composition comprising a food-grade carrier.

114. The method of claim 102, wherein the polyphenol is administered in a functional food composition comprising a food-grade carrier.

115. The method of claim 103, wherein the polyphenol is encapsulated in a liposome, nanoparticle, or microsphere.

116. A method of treating a subject, comprising:
ascertaining a microbiota index measured in a sample of subgingival fluid from the individual;
comparing the microbiota index to a reference value determined through statistical analysis or machine learning of the preponderance of microbial genera or species in subgingival fluid from healthy and diseased subjects;
determining the degree of oral dysbiosis in the individual based on the comparison; and
administering a composition to the subject until the degree of oral dysbiosis is within a predetermined range;
wherein the composition comprises a polyphenol and a material which induces CAMP gene expression in the individual.

117. The method of claim 116, wherein the microbiota index is ascertained by performing a DNA sequencing analysis on the sample of subgingival fluid.

118. The method of claim 116, wherein the reference value is determined through machine learning algorithms trained on a dataset comprising microbiota information from a plurality of healthy and diseased subjects.

119. The method of claim 116, wherein the degree of oral dysbiosis is determined by calculating a dysbiosis index based on the comparison of the microbiota index and the reference value.

120. The method of claim 116, wherein the composition further comprises an antimicrobial agent selected from the group consisting of chlorhexidine, cetylpyridinium chloride, and triclosan.

121. The method of claim 116, wherein the composition further comprises a probiotic microorganism selected from the group consisting of Lactobacillus acidophilus, Bifidobacterium bifidum, and Streptococcus thermophilus.

122. The method of claim 116, wherein the composition is administered to the subject via oral application.

123. The method of claim 116, wherein the predetermined range of oral dysbiosis is determined based on the subject's medical history, age, gender, and lifestyle factors.

124. The method of claim 116, wherein the composition is administered to the subject in conjunction with a dental procedure selected from the group consisting of scaling, root planing, and periodontal surgery.

125. The method of any one of claims 116-124, wherein the polyphenol is selected from the group consisting of epigallocatechin-3-gallate (EGCG), resveratrol, quercetin, and curcumin.

126. The method of any one of claims 116-125, wherein the material which induces CAMP gene expression is selected from the group consisting of vitamin D, butyrate, and β-glucan.

127. The method of any one of claims 116-126, wherein the composition is administered to the subject in a sustained-release form.

128. The method of any one of claims 116-127, wherein the composition is administered to the subject in a combination therapy with an antibiotic.

129. The method of any one of claims 116-128, wherein the subject has a history of periodontal disease.

130. The method of any one of claims 116-129, wherein the degree of oral dysbiosis is monitored over time to adjust the dosage and duration of the composition administration.

131. The method of any one of claims 116-130, wherein the composition is administered to the subject in combination with an oral hygiene regimen consisting of tooth brushing and flossing.

132. A method of treating a subject, comprising:
ascertaining a microbiota index measured in a sample taken from a source microbiome selected from the group consisting of the gut, skin, urinary, aural, ocular, genital, pulmonary, nasopharyngeal, tonsillar, and umbilical microbiomes;
comparing the microbiota index to a reference value determined through statistical analysis or machine learning of the preponderance of microbial genera or species in a sample from healthy and diseased subjects in the source microbiome;
determining the degree of dysbiosis in the source microbiome based on the comparison; and
administering a composition to the subject until the degree of dysbiosis in the source microbiome is within a predetermined range;
wherein the composition comprises a polyphenol and a material which induces CAMP gene expression in the individual.

133. The method of claim 132, wherein the ocular microbiome includes microorganisms inhabiting the surface of the eye and surrounding structures, including the conjunctiva, eyelids, and tear ducts.

134. The method of claim 132, wherein the microbiota index is ascertained by performing a DNA sequencing analysis on the sample.

135. The method of claim 132, wherein the reference value is determined through machine learning algorithms trained on a dataset comprising microbiota information from a plurality of healthy and diseased subjects.

136. The method of claim 132, wherein the degree of dysbiosis is determined by calculating a dysbiosis index based on the comparison of the microbiota index and the reference value.

137. The method of claim 132, wherein the composition further comprises an antimicrobial agent selected from the group consisting of chlorhexidine, cetylpyridinium chloride, and triclosan.

138. The method of claim 132, wherein the composition further comprises a probiotic microorganism selected from the group consisting of Lactobacillus acidophilus, Bifidobacterium bifidum, and Streptococcus thermophilus.

139. The method of claim 132, wherein the composition is administered to the subject via oral application.

140. The method of claim 132, wherein the composition is administered to the subject via topical application.

141. The method of claim 132, wherein the composition is administered to the subject via transdermal application.

142. The method of claim 132, wherein the predetermined range of dysbiosis is determined based on the subject's medical history, age, gender, and lifestyle factors.

143. The method of any one of claims 132-142, wherein the polyphenol is selected from the group consisting of epigallocatechin-3-gallate (EGCG), resveratrol, quercetin, and curcumin.

144. The method of any one of claims 132-143, wherein the material which induces CAMP gene expression is selected from the group consisting of vitamin D, butyrate, and β-glucan.

145. The method of any one of claims 132-144, wherein the composition is administered to the subject in a sustained-release form.

146. The method of any one of claims 132-145, wherein the composition is administered to the subject in a combination therapy with an antibiotic.

147. The method of any one of claims 132-146, wherein the degree of oral dysbiosis is monitored over time to adjust the dosage and duration of the composition administration.

148. The method of any one of claims 132-147, wherein ascertaining a microbiota index measured in a sample taken from a source microbiome includes ascertaining a first microbiota index measured in a sample taken from a first source microbiome and ascertaining a second microbiota index measured in a sample taken from a second source microbiome, wherein said first and second source microbiomes are distinct.

149. The method of claim 148, wherein comparing the microbiota index to a reference value includes comparing the first microbiota index to a first reference value and comparing the second microbiota index to a second reference value.

150. The method of claim 149, wherein determining the degree of dysbiosis includes performing multivariate analysis based on the first and second microbiota indices.

151. The method of claim 150, wherein determining the degree of dysbiosis includes performing multivariate analysis based on the first and second reference values.
